(19) United States
(12) Patent Application Publication
(10) Pub. No.: US 2025/0215480 A1
(43) Pub. Date: Jul. 3, 2025

(54) METHODS AND SYSTEMS FOR DIGITAL MULTIPLEX ANALYSIS

(71) Applicant: ChromaCode, Inc.

(72) Inventor: Christopher MACDONALD, Carlsbad, CA (US)

(21) Appl. No.: 19/011,189

(22) Filed: Jan. 6, 2025

Related U.S. Application Data
(63) Continuation of application No. PCT/US2023/069722, filed on Jul. 6, 2023.
(60) Provisional application No. 63/367,852, filed on Jul. 7, 2022.
(60) Provisional application No. 63/367,865, filed on Jul. 7, 2022.

(51) Int. Cl. C12Q 1/682; C12Q 1/6825; C12Q 1/6851; C12Q 1/686

(52) U.S. Cl. CPC C12Q 1/682; C12Q 1/6825; C12Q 1/6851; C12Q 1/686

(57) ABSTRACT

The present disclosure provides methods, systems, and compositions for the multiplexed detection and quantification of multiple analytes from a sample. Analytes may be nucleic acid analytes. Detection of analytes may comprise contacting one or more sample subsets with hybridization probes to generate cumulative signal measurements. The methods may comprise digital PCR or may comprise partitioning a sample into multiple partitions.

### CROSS-REFERENCE

This application is a continuation of International Patent Application No. PCT/US2023/069722 filed Jul. 6, 2023, which claims benefit and priority to U.S. Provisional Patent Application No. 63/367,852 filed Jul. 7, 2022 and U.S. Provisional Patent Application No. 63/367,865 filed Jul. 7, 2022, each of which are incorporated by reference herein in their entirety.

### BACKGROUND

Digital PCR (dPCR) is a useful method for detection and quantification of nucleic acid targets. The use of labeled oligonucleotide probes enables specific detection of a target present in a partition (e.g., droplet, microwell). dPCR methods may involve creating spatially resolved clusters of fluorescent points and drawing a cutoff or threshold between the clusters to identify whether an individual partition contains a target of interest.

### SUMMARY

Disclosed herein, in some aspects, are methods for multiplex quantitation wherein individual partitions need not be classified to quantify the concentration of targets present in the reaction.

Digital amplification reaction detection systems present an opportunity to enhance many of the capabilities of these systems to detect analytes including increased sensitivity, selectivity, robustness, and throughput. However, paired with these potential benefits, new technological challenges are also presented by such detection systems. The methods described herein provide analytic solutions to some of the problems inherent to digital amplification assays.

The systems and method described herein may allow for users of amplification reaction detection systems to compensate for inconsistency in sample preparation or limitations of sample types. For example, a poorly diluted sample may change the expected number of partitions that contain more than one target. Additionally, biological samples may be limited in volume and of variable composition so that it is difficult to optimize the concentration of the template. In these examples the methods and systems described herein may allow the detection or quantification assay to be performed with problematic samples without detrimentally effecting the quality of the results.

In some aspects of the methods and systems provided herein, is a method of quantifying a first nucleic acid target and a second nucleic acid target in a sample is provided. The method may comprise providing a mixture comprising which comprises a first nucleic acid target, a second nucleic acid target, a first plurality of oligonucleotide probes, hybridizes to the first nucleic acid target, and a second plurality of oligonucleotide probes, which hybridizes to the second nucleic acid target. In some embodiments, the method may comprise partitioning said mixture into a plurality of partitions. In some embodiments, the method may comprise in a plurality of partitions, generating a plurality of signals from a first plurality of probes and a second plurality of probes. In some embodiments, the plurality of signals may be detectable in one or more color channels. In some embodiments, the method may comprise detecting from multiple partitions a plurality of signals in said one or more color channels. In some embodiments, the method may comprise quantifying a first nucleic acid target and a second nucleic acid target in a sample, in which the quantification does not comprise using a determined number of partitions positive for two or more of said targets.

In some embodiments, the method may comprise generating at least 10 partitions positive for two or more of said targets. In some embodiments, the method may comprise generating at least 100 partitions positive for two or more of said targets. In some embodiments, the method may comprise generating a number of partitions positive for two or more targets that is greater than 10% of the total number of partitions. In some embodiments, the method may comprise generating a number of partitions comprising positive for two or more targets that is greater than 20% of the total number of partitions. In some embodiments, the quantification may comprises determining a total number of partitions. In some embodiments, quantification is performed without determining a total number of partitions.

In some embodiments, the first or second plurality of probes may comprise a fluorophore. In some embodiments, a plurality of signals may be generated by excitation of a fluorophore. In some embodiments, a plurality of signals may be generated by degradation of a first or second plurality of oligonucleotide probes.

In some embodiments, the method may comprise amplifying a plurality of nucleic acid molecules, thereby generating said plurality of signals. In some embodiments, the method may comprise amplification comprising a polymerase chain reaction. In some embodiments, the method may comprise amplification comprises an isothermal amplification reaction.

In some embodiments, a sample may be derived from a biological sample. In some embodiments, a biological sample may be blood or plasma. In some embodiments, a biological sample may be a maternal plasma sample. In some embodiments, a biological sample may be a plasma sample from an organ transplant recipient. In some embodiments, a mixture may be generated by diluting a biological sample by no more than 10%. In some embodiments, a mixture may be generated by diluting said biological sample by no more than 20%. In some embodiments, a mixture may be generated by diluting said biological sample by no more than 50%. In some embodiments, a biological sample may not be diluted prior to addition to said mixture. In some embodiments, a first nucleic acid target or a second nucleic acid target may comprise a chromosomal nucleic acid sequence. In some embodiments, a chromosomal nucleic acid may be a sequence may be chosen from among the nucleic acid sequences of chromosome 21, chromosome 18, chromosome 15, chromosome 13, an X chromosome, or a Y chromosome. In some embodiments, a first nucleic acid target may comprise a first chromosomal nucleic acid sequence and said second nucleic acid target may comprise a second chromosomal nucleic acid sequence. In some embodiments, a first nucleic acid target or second nucleic target may be derived from a virus or bacteria. In some embodiments, a first nucleic acid target or second nucleic target may comprise RNA. In some embodiments, a first nucleic acid target or second nucleic target may comprise cDNA. In some embodiments, a first nucleic acid target or second nucleic target may be processed to generate cDNA. In some embodiments, a first nucleic acid target or second nucleic target may comprise nucleic acids derived from a fetus. In some embodiments, a first nucleic acid target may be derived from a first organism and said second nucleic target may be derived from a second organism. In some embodiments, a first nucleic acid target may be derived from a first individual and a second nucleic target may be derived from a second individual.

In some embodiments, a plurality of signals may be a plurality of fluorescent signals or a plurality of chemiluminescent signals. In some embodiments, quantification may comprise an absolute quantification of a first nucleic acid target or a second nucleic acid target. In some embodiments, quantification may comprise a relative quantification of a first nucleic acid target or a second nucleic acid target. In some embodiments, quantification may comprise generating an average number of nucleic acid molecules per partition. In some embodiments, quantification may comprise generating a probability that one or more nucleic acid molecules of a first or a second plurality of nucleic acid molecules is present in a given partition. In some embodiments, quantification may comprise, for a plurality of partitions, generating a probability that one or more nucleic acid molecules of a first or a second plurality of nucleic acid molecules is present in a given partition of a plurality of partitions, thereby generating a plurality of probabilities. In some embodiments, quantification may comprise summing two or more probabilities of a plurality of probabilities. In some embodiments, quantification may comprise determining a total number of partitions. In some embodiments, quantification may be performed without determining a total number of partitions. In some embodiments, a plurality of partitions may comprise a plurality of droplets.

In some aspects of the methods and systems provided herein, a method of quantifying at least a first nucleic acid in a target in a sample is provided. In some embodiments, the method may comprise a mixture comprising a first target nucleic acid target, a second target nucleic acid target, a third target nucleic acid target. In some embodiments, the method may comprise a mixture comprising a first plurality of oligonucleotide probes which hybridizes to a first nucleic acid target, a second plurality of oligonucleotide probes which hybridizes to a second nucleic acid target, and a third plurality of oligonucleotide probes, which hybridizes to a third nucleic acid target. In some embodiments, the method may comprise partitioning said mixture into a plurality of partitions. In some embodiments, the method may comprise, in a plurality of partitions, generating a plurality of signals from a first plurality of probes, a second plurality of probes and a third plurality of probes. In some embodiments, a plurality of signals may be detectable in one or more color channels. In some embodiments, the method may comprise detecting from multiple partitions of a plurality of partitions a plurality of signals in one or more color channels. In some embodiments, the method may comprise a plurality of signals comprising an indifferentiable signal. In some embodiments, the indifferentiable signal may be generated from at least one partition of the plurality of partitions which may comprise a first target nucleic acid target, a second target nucleic acid target, a third target nucleic acid target, or a combination thereof. In some embodiments, the method may comprise quantifying a first nucleic acid target in a sample. In some embodiments, quantification does not comprise using a determined number of partitions positive for a indifferentiable signal generated from said two or more other targets.

In some embodiments, the method may comprise generating at least 10 partitions that generate an ambiguous signal. In some embodiments, the method may comprise generating at least 100 partitions that generate an ambiguous signal. In some embodiments, the method may comprise generating a number of partitions that generate an ambiguous signal that is greater than 10% of the total number of partitions. In some embodiments, the method may comprise generating a number of partitions that generate an ambiguous signal that is greater than 20% of the total number of partitions. In some embodiments, the first, second, and/or third plurality of probes may comprise a fluorophore. In some embodiments, a plurality of signals may be generated by excitation of one or more fluorophores. In some embodiments, a plurality of signals may be generated by degradation of a first, second, and/or third plurality of oligonucleotide probes.

In some embodiments, the method comprises amplifying a plurality of nucleic acid molecules, thereby generating said plurality of signals. In some embodiments, the method may comprise an amplification comprising a polymerase chain reaction. In some embodiments, the method may comprise an amplification comprises an isothermal amplification reaction.

In some embodiments, a sample may be derived from a biological sample. In some embodiments, a biological sample may be blood or plasma. In some embodiments, a biological sample may be a maternal plasma sample. In some embodiments, a biological sample may be a plasma sample from an organ transplant recipient. In some embodiments, a mixture may be generated by diluting a biological sample by no more than 10%. In some embodiments, a mixture may be generated by diluting said biological sample by no more than 20%. In some embodiments, a mixture may be generated by diluting said biological sample by no more than 50%. In some embodiments, a biological sample may not be diluted prior to addition to said mixture.

In some embodiments, a first target nucleic acid target, a second target nucleic acid target, and/or a third target nucleic acid target may comprise a chromosomal nucleic acid sequence. In some embodiments, a chromosomal nucleic acid sequence may be chosen from among the nucleic acid sequences of chromosome 21, chromosome 18, chromosome 15, chromosome 13, an X chromosome, or a Y chromosome. In some embodiments, a first target nucleic acid target comprises a first chromosomal nucleic acid sequence, a second target nucleic acid target comprises a second chromosomal nucleic acid sequence, and a third target nucleic acid target comprises a third chromosomal nucleic acid sequence. In some embodiments, a first target nucleic acid target, a second target nucleic acid target, and/or a third target nucleic acid target may be derived from a virus or bacteria. In some embodiments, a first target nucleic acid target, a second target nucleic acid target, and/or a third target nucleic acid target may comprise RNA. In some embodiments, a first target nucleic acid target, a second target nucleic acid target, and/or a third target nucleic acid target may comprise cDNA. In some embodiments, a first target nucleic acid target, a second target nucleic acid target, and/or a third target nucleic acid target may be processed to generate cDNA. In some embodiments, a first target nucleic acid target, a second target nucleic acid target, and/or a third target nucleic acid target may comprise nucleic acids derived from a fetus. In some embodiments, any one or more of a first target nucleic acid target, a second target nucleic acid, and/or a third target nucleic acid may be derived from a first organism. In some embodiments, any one or more of a first target nucleic acid target, a second target nucleic acid, and/or a third target nucleic acid may be derived from a second organism. In some embodiments, any one or more of a first target nucleic acid target, a second target nucleic acid, and/or a third target nucleic acid may be derived from a first individual. In some embodiments, any one or more of a first target nucleic acid target, a second target nucleic acid, and/or a third target nucleic acid may be derived from a second individual.

In some embodiments, a plurality of signals may be a plurality of fluorescent signals or a plurality of chemiluminescent signals. In some embodiments, a indifferentiable signal may be generated from a partition comprising two or more of a first nucleic acid target, a second target nucleic acid and a third target nucleic acid. In some embodiments, quantification may comprise an absolute quantification of a first target nucleic acid target, a second target nucleic acid target, or a third target nucleic acid target. In some embodiments, quantification may comprise an relative quantification of a first target nucleic acid target, a second target nucleic acid target, or a third target nucleic acid target. In some embodiments, quantification may comprise generating an average number of nucleic acid molecules per partition. In some embodiments, quantification may comprise generating a probability that one or more nucleic acid molecules of a first target nucleic acid target, a second target nucleic acid target, or a third target nucleic acid target is present in a given partition. In some embodiments, quantification may comprise, for said plurality of partitions, generating a probability that one or more nucleic acid molecules of a first target nucleic acid target, a second target nucleic acid target, or a third target nucleic acid target is present in a given partition of said plurality of partitions, thereby generating a plurality of probabilities. In some embodiments, quantification may comprise summing two or more probabilities of a plurality of probabilities. In some embodiments, quantification may comprise determining a total number of partitions. In some embodiments, quantification may be performed without determining a total number of partitions.

In some embodiments, the plurality of partitions may comprise a plurality of droplets. In some embodiments, the method may comprise quantifying two nucleic acid targets in a single channel with two resolvable levels. In some embodiments, the method may comprise quantifying three nucleic acid targets in a single channel with three resolvable levels. In some embodiments, the method may comprise quantifying four nucleic acid targets in a single channel with four resolvable levels. In some embodiments, the method may comprise quantifying X nucleic acid targets in a single channel with X resolvable levels. In some embodiments, the method may comprise quantifying two more nucleic acid targets in a second channel comprising two resolvable levels. In some embodiments, the method may comprise quantifying three more nucleic acid targets in a second channel comprising three resolvable levels. In some embodiments, the method may comprise quantifying four more nucleic acid targets in a second channel comprising four resolvable levels. In some embodiments, the method may comprise quantifying Y more nucleic acid targets in a second channel comprising Y resolvable levels.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an example plot of intensity with labeled peaks: N0: Number of partitions with no targets; N1: Number of partitions with target 1; N2: Number of partitions with target 2; N3: Number of partitions with both target 1 and target 2.

FIG. 2 illustrates an example method for quantifying the number of target nucleic acids in a sample without utilizing partitions that contain more than one target nucleic acid.

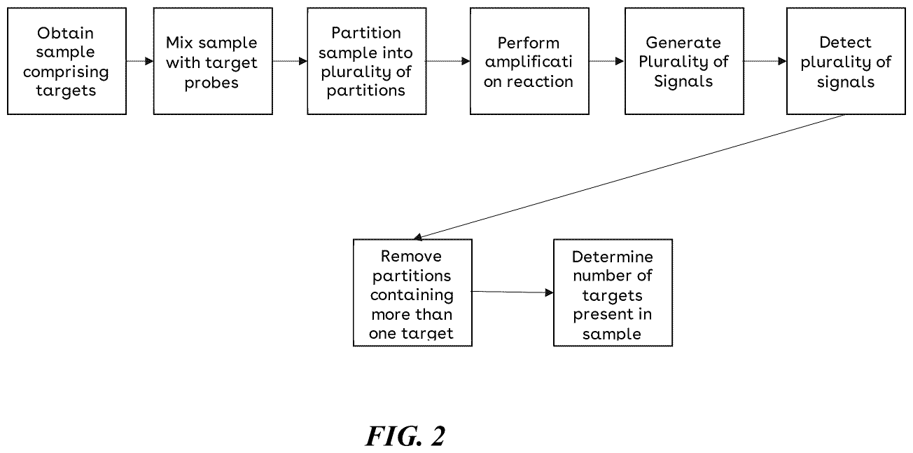

FIG. 3 illustrates an example method for quantifying the number of target nucleic acids in a sample containing three or more target nucleic acids without utilizing partitions that have an ambiguous composition of target nucleic acids.

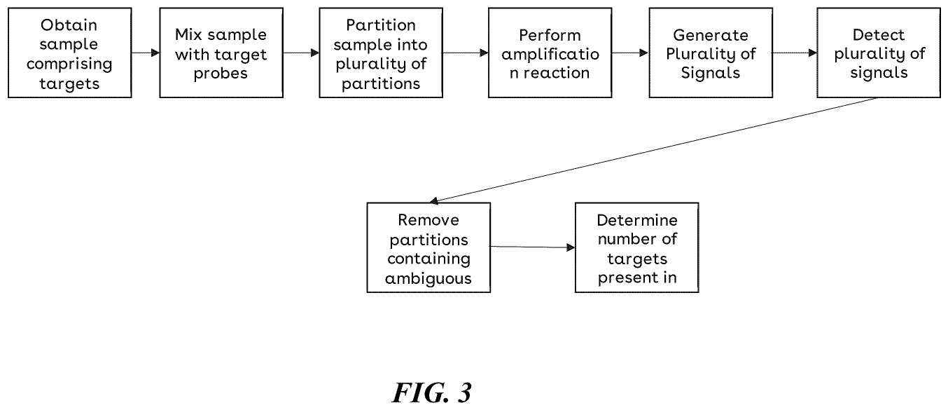

FIG. 4A illustrates the ability to target multiple loci on a single chromosome with a digital PCR assay as shown in a single channel of droplet intensities. FIG. 4B illustrates the ability to target multiple loci on a single chromosome with a digital PCR assay as shown in a single channel of fluorescent intensity versus partition count.

FIG. 5A illustrates the ability to target multiple loci on a single chromosome with a digital PCR assay as shown in a single channel of droplet intensities. FIG. 5B illustrates the ability to target multiple loci on a single chromosome with a digital PCR assay as shown in a single channel of fluorescent intensity versus partition count.

### DETAILED DESCRIPTION

The following description provides specific details for a comprehensive understanding of, and enabling description for, various embodiments of the technology. It is intended that the terminology used be interpreted in its broadest reasonable manner, even where it is being used in conjunction with a detailed description of certain embodiments.

Before describing the present teachings in detail, it is to be understood that the disclosure is not limited to specific compositions or process steps, and as such, may vary. As used herein, the singular forms “a,” “an,” and “the” are intended to include the plural forms as well, unless the context clearly indicates otherwise. Furthermore, to the extent that the terms “including,” “includes,” “having,” “has,” “with,” “such as,” or variants thereof, are used in either the specification and/or the claims, such terms are not limiting and are intended to be inclusive in a manner similar to the term “comprising.” Unless specifically noted, embodiments in the specification that recite “comprising” various components are also contemplated as “consisting of” or “consisting essentially of” the recited components.

Polymerase Chain Reaction (PCR) is a method of exponential amplification of specific nucleic acid target in a reaction mix with a nucleic acid polymerase and primers. Primers are short single stranded oligonucleotides which are complementary to the 3′ sequences of the positive and negative strand of the target sequence. The reaction mix is cycled in repeated heating and cooling steps. The heating cycle denatures or splits a double stranded nucleic acid target into single stranded templates. In the cooling cycle, the primers bind to a complementary sequence on the template. After the template is primed the nucleic acid polymerase creates a copy of the original template. Repeated cycling exponentially amplifies the target 2-fold with each cycle leading to approximately a billion-fold increase of the target sequence in 30 cycles (Saiki et al 1988).

Digital PCR (dPCR) is a process of partitioning a sample containing one or more targets into a plurality of partitions (e.g., wells, droplets, etc.), performing a PCR reaction in each partition, and recording the luminescence (e.g., fluorescence) generated by, for example, a target-specific reporter probe. The use of labeled oligonucleotide probes enables specific detection. dPCR may be used in a variety of nucleic acid detection methods. Digital PCR is generally performed on a digital PCR instrument that measures the fluorescence from each partition in an optical channel through one or more excitation/emission filter sets.

Frequently, the target-specific oligonucleotide probe is a short oligonucleotide complementary to one strand of the amplified target. The probe lacks a 3′ hydroxyl and therefore is not extendable by the DNA polymerase. TaqMan® (ThermoFisher Scientific) chemistry is a common reporter probe method used for multiplex Real-Time PCR (Holland et al. 1991). The TaqMan® oligonucleotide probe is covalently modified with a fluorophore and a quenching tag (i.e., quencher). In this configuration the fluorescence generated by the fluorophore is quenched and is not detected by the real time PCR instrument. When the target of interest is present, the probe oligonucleotide base pairs with the amplified target. While bound, it is digested by the 5′ to 3′ exonuclease activity of the Taq polymerase thereby physically separating the fluorophore from the quencher and liberating signal for detection by the real time PCR instrument.

Multiplex analysis of multiple nucleic acid targets in a single measurement may be performed by encoding each nucleic acid target to a unique intensity value or range of values. For example, for detection of multiple nucleic acid targets in a sample using a single measurement, oligonucleotide probes may be provided at varying concentrations, such that the intensity of each signal generated from the probes, both individually and in combination, is unique.

### Overview

In one example of dPCR, a single sample containing at least one nucleic acid target sequence, at least one amplification oligomer, at least one detection oligonucleotide, dNTPs, a DNA polymerase, and other PCR reagents may be partitioned into approximately 20,000 evenly sized partitions. Generally, each partition may receive a single template of the nucleic acid target sequence. However, statistically, some partitions may receive more than one copy of a nucleic acid target template, while other partitions may not receive any target template.

Following partitioning, each partition may be subject to end-point PCR. Partitions emitting a fluorescent signal are marked “positive” and scored as “1,” whereas partitions without detectable fluorescence are deemed “negative” and scored as “0.” The underlining theory of dPCR is that the number of positive reactions is directly proportional to the total number of template nucleic acid present in the sample—thus enabling absolute quantification. However, because some partitions will receive more than one nucleic acid target template during partitioning, if uncorrected, the proportion of positive partitions will not accurately reflect the precise quantity of nucleic acid target present in the sample. Thus, a Poisson statistical model may be used to calculate the probability of a given reaction receiving zero, one, two, three or more copies. This “correction” may enable all molecules in the starting sample to be accounted for, yielding absolute quantification. For a single target amplification, the quantity of nucleic acid in terms of average number of copies per partition (λ) can be determined by λ=−ln(P(k=0)).

Described herein are methods and systems that may allow for the absolute quantification of targets in a sample without having to use partitions that have more than one target.

When a sample is partitioned, there is a possibility that more than one target nucleic acid may be present within a single partition. This likelihood increases as the concentration of the targets is increased. This may complicate the analysis or limit the sensitivity of the assay due to uncertainty associated with the inability to determine contents of the partitions containing multiple targets. However, certain embodiments of the method and systems herein allow for the ability to obtain high sensitivity without requiring a means to determine the contents of partitions containing more than one target.

In another example, the methods may be used to quantify the two separate targets. Once data is received from a digital PCR run and identification is performed on which point cluster each droplet falls into, the final step in producing an answer is to map these cluster counts back to a set of target concentrations. In general, targets distribute themselves according to the Poisson distribution, where k for each target corresponds to the number of counts of that target divided by the number of droplets. However, the situation is complicated a bit by the fact that one cannot directly observe how many copies of a given target are present in a drop (“k”); so it is generally only possible to determine whether k=0 or k>0. For a given ratio, the Poisson probabilities of these two states are:

p(k=0):exp(−λ)

p(k>0):1−exp(−λ)

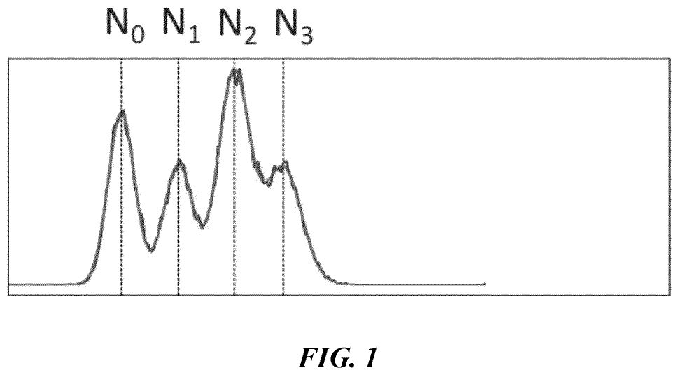

If there is only one single target, it is trivial to solve for the unique value of lambda that solves these equations; it is simply λ=−ln(p(k=0)) or λ=−ln(1−p(k>0)). If there are two targets coded at different intensity levels, they can be calculated independently. Assume we have two targets with droplet fraction occupancy p1=1−exp(−λ1) at level 1 and p2=1−exp(−λ2) at level 2. Also assume that the probability of presence of the targets in a given partition are independent of each other, and the number of partitions at each level are N0 . . . N3. FIG. 1 shows an example plot of intensity with labeled peaks:

N0: Number of partitions with no targets; N1: Number of partitions with target 1; N2: Number of partitions with target 2; N3: Number of partitions with both target 1 and target 2.

Since the probability of occupancy of a given partition with target 1 (p1) is independent of occupancy with target 2 (p2), then the ratio of partitions with target 1 in the subset of partitions without target 2 would be

$${p}_{1} = \frac{{N}_{1}}{{N}_{0} + {N}_{1}}$$

and the partitions containing target 2 (N2, N3) would also lead to

$$p 1 = N 3 / ( N 2 + N 3 ) \qquad {p}_{1} = \frac{{N}_{3}}{{N}_{2} + {N}_{3}}$$

And across all partitions,

$${p}_{1} = \frac{{N}_{1} + {N}_{3}}{\sum N}$$

Thus, the calculation of the concentration of target 1 in number of average copies per partition (λ1) is

$${λ}_{1} = - ln ( 1 - {p}_{1} ) = - ln ( 1 - \frac{{N}_{1}}{{N}_{0} + {N}_{1}} ) = - ln ( 1 - \frac{{N}_{3}}{{N}_{2} + {N}_{3}} ) = - ln ( 1 - \frac{{N}_{1} + {N}_{3}}{\sum N} )$$

This results in the ability to calculate the concentration of the targets in the above multiplexed reaction without using the number or count of partitions of any partition with two or more targets present in them (e.g. N3 is not needed). The concentration of the second target can be calculated with the same method,

$${λ}_{2} = - ln ( 1 - {p}_{2} ) = - ln ( 1 + \frac{{N}_{2}}{{N}_{0} + {N}_{2}} )$$

The plurality of signals may be generated by one or more of the plurality of probes from the mixture. The plurality of signals may be generated by nucleic acid amplification (e.g., PCR) of the plurality of nucleic acid molecules. Nucleic acid amplification may degrade the plurality of oligonucleotide probes (e.g., by activity of a nucleic acid enzyme), thereby generating the plurality of signals. A plurality of signals may be a plurality of fluorescent signals, a plurality of chemiluminescent signals, or a combination thereof.

In multiple aspects as described herein, signals and data relating to the detection of the signals are subjected to processing in order for the signals and data to be used for subsequent steps or downstream methods. The processing may use mathematical algorithms to analyze or process the signal data. In some case, the processing may use data obtained from the instrument or detector. The processing may use data obtained from multiple channels, or a single channel. In some cases, the processing may use data from channels that are not expected to correlate with a signal from a given probe or fluorophore. For example, the data may include data obtained from a reference channel in which a background signal is obtained. The processing may use data obtained from all available channels of a given detection device.

Described herein are methods and systems that may allow for the absolute quantification of multiple targets in a sample without using partitions that have ambiguous signals.

When sample is partitioned, there is a possibility that more than one target nucleic acid may be present within a single partition. This may make the identification of the targets present in each partition difficult or impossible. This may be because the signal generated from the partition may be ambiguous. This ambiguity may be present because the signal generated from one or more targets may be indistinguishable from a signal generated from a partition containing a different one or more targets. However, certain embodiments of the methods and systems herein may allow for a assay that is able to obtain high sensitivity without requiring a means to determine the contents of partitions that are generating ambiguous signals. This may allow for higher levels of multiplexing within the same sample.

In another example, the methods may be used to quantify multiple targets. The methods may allow for quantifying targets independent of partitions positive for two or more targets, where targets are encoded in such a way that intensity levels could be representative of multiple target combinations. For example, consider a system containing three targets of concentrations per droplet λ1, λ2, and λ3, and thus a probability of a partition being occupied by at least one target of pn=exp(−λn) and fraction occupancies N0 . . . N6 where
N0: Number of partitions with no targets
N1: Number of partitions with target 1
N2: Number of partitions with target 2
N3: Number of partitions with both target 1 and target 2 OR only target 3
N4: Number of partitions with target 1 and target 3
N5: Number of partitions with target 2 and target 3
N6: Number of partitions with both target 1 and target 2 and target 3

In this situation, the original calculations for concentrations of targets 1 and 2 apply using a subset of partitions,

$${λ}_{1} = - ln ( 1 - {p}_{1} ) = - ln ( 1 - \frac{{N}_{1}}{{N}_{0} + {N}_{1}} )$$

$${λ}_{2} = - ln ( 1 - {p}_{2} ) = - ln ( 1 - \frac{{N}_{2}}{{N}_{0} + {N}_{2}} )$$

However, since N3 consists of both partitions positive for targets 1 and 2 OR only target 3, the concentration of target 3 cannot be calculated the same way. In order to do the correct concentration calculation, the total amount of partitions positive for target 3 only must be known.

Let Nt3 be the number of partitions positive for target 3 only, and Nt1+t2 be the number of partitions positive for both targets 1 and 2 but not target 3. In this case,

$${λ}_{3} = - ln ( 1 - {p}_{3} ) = - ln ( 1 - \frac{{N}_{t 3}}{{N}_{0} + {N}_{t 3}} ) , {N}_{3} = {N}_{t 3} + {N}_{t 1 + t 2}$$

$$And$$

$${N}_{t 3} = {N}_{3} - {N}_{t 1 + t 2}$$

Let N˜3 be all partitions that do not have target 3, or

$${N}_{\sim 3} = {N}_{0} + {N}_{1} + {N}_{2} + ( {N}_{3} - {N}_{t 3} )$$

And then the expected number of partitions containing both targets 1 and 2 but not three can be calculated as

$${N}_{t 1 + t 2} = {p}_{1} {p}_{2} {N}_{\sim 3}$$

And then

$${N}_{t 3} = {N}_{3} - {p}_{1} {p}_{2} {N}_{\sim 3} = {N}_{3} - {p}_{1} {p}_{2} ( {N}_{0} + {N}_{1} + {N}_{2} + ( {N}_{3} - {N}_{t 3} ) )$$

Substituting the equations for p1 and p2 above and solving for Nt3 reduces to

$${N}_{t 3} = {N}_{3} = - \frac{{N}_{2} {N}_{1}}{{N}_{0}}$$

And so, the concentration of target 3 can be estimated directly from only partitions positive for zero or one targets while the true count of partitions positive for any combination of targets (e.g. Nt1+t2) is unknown and not needed,

$${λ}_{3} = - ln ( 1 - {p}_{3} ) = - ln ( 1 - \frac{{N}_{t 3}}{{N}_{0} + {N}_{t 3}} ) = - ln ( 1 - \frac{{N}_{0} {N}_{3} - {N}_{1} {N}_{2}}{{{N}_{0}}^{2} + {N}_{0} {N}_{3} - {N}_{1} {N}_{2}} )$$

With this method, all three concentrations can be directly calculated from a subset of the partitions in the reaction even with some unknown values and inability to count combinations of targets. This resolves a critical problem identified in the literature where multiplexing was limited to input concentrations so low that the number of partitions with targets 1 and 2 being present (Nt1+t2) is effectively zero, and an issue where the concentration of target 3 was not calculable and requires the signals for (target 1+target 2) and (target 3) to be differentiable and independently countable.

The advantage of this method over existing methods is shown in the above example. The number of targets that can be multiplexed in a given channel for N resolvable intensity levels above zero is log 2(N+1) for previous methods (e.g. for four targets, must be encoded at levels 1, 2, 4, 8 for 15 levels to generate unique combinations. In the case of the method above, one target can be encoded per intensity level with the expected values of target combinations used to determine the number of a given target present without needing to determine any number of partitions that have more than one target present in them. This method allows encoding one target per intensity level, so for N resolvable levels above zero, N targets can be encoded.

| Number of resolvable | Max targets (from | Max Targets (Methods |
|---|---|---|
| levels | literature) | disclosed herein) |
| 1 | 1 | 1 |
| 2 | 1 | 2 |
| 3 | 2 | 3 |
| 4 | 2 | 4 |
| 5 | 2 | 5 |
| 6 | 2 | 6 |
| 7 | 3 | 7 |
| 8 | 3 | 8 |
| 9 | 3 | 9 |

When the result of amplification of each partition is visualized as a two-dimensional scatter plot (whereby fluorescent amplitudes in two color channels are plotted against each other), the resulting fluorescent “clusters” may be indicative of the relative amount of the specific nucleic acid target sequence in the sample results.

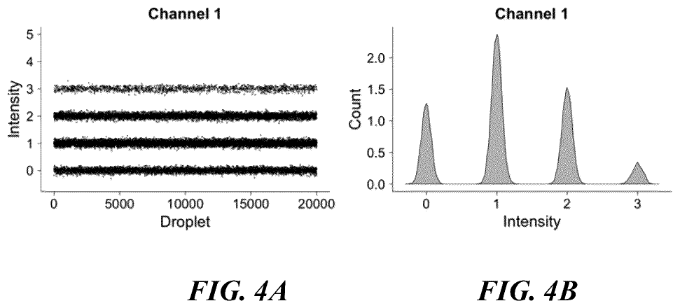

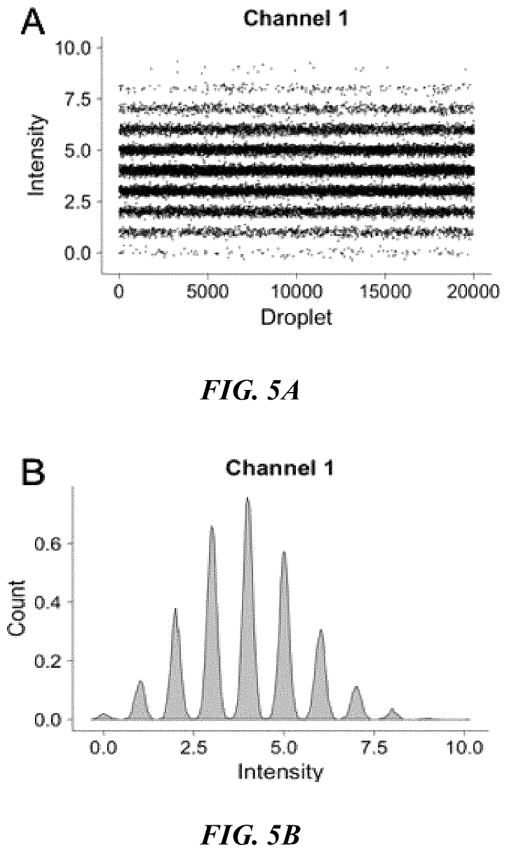

Resolvable levels mean intensity levels within a single channel that can be identified as separate populations. For example, FIG. 4A demonstrates four resolvable levels corresponding to an intensity level of 0, 1, 2, and 3. There may be more resolvable levels. For example, FIG. 5A demonstrates 10 discrete resolvable levels. The populations at each intensity level may comprise a single plurality of partition corresponding to a single copy of a one nucleic acid target. The populations at each intensity level may comprise a single plurality of partition corresponding to a more than one copy of a one nucleic acid target. The populations at each intensity level may comprise multiple pluralities of portions corresponding to different nucleic acid targets. Each plurality of partitions may comprise a single copy or multiple copies of each of one or more nucleic acid targets. The composition of the plurality of partitions may or may not be determinable based upon the intensity level of the of the plurality of partitions.

The method may use 1, 2, 3, 4, 5, 6, 7, 8, 9 or more resolvable levels. One or more channels may have 1, 2, 3, 4, 5, 6, 7, 8, 9, or more resolvable levels. Different channels may have a different number of resolvable levels. The maximum number of detectable targets in a single channel may depend on the number of resolvable levels in that channel. The number of detectable targets in a single channel may be the same as the number of resolvable levels.

When multiplexing multiple targets into two channels of a digital PCR reaction, there may be overlap between many of the final states. For example, in an experiment where each detection probe utilizes a reporter dye (e.g., FAM and HEX), when plotted, the clusters will comprise HEX-positive, FAM-positive, double-positive, and double-negative (empty) clusters. However, in practice, not all partitions provide definitively positive, or negative, results. Ambiguous partitions are generally observed between clusters on a two-dimensional scatter plot. Ambiguous signals may be present between clusters in the same color channel. Ambiguous signals may be present between clusters in different color channels. The presence of ambiguous signals, falling between distinctly positive and distinctly negative populations, may prevent precise quantification. Recognized herein is a need for quantification methods which account for ambiguous signals.

In some aspects, the present disclosure provides methods, systems, and compositions for multiplex quantification using digital assays, wherein individual partitions need not be classified to quantify targets present in a reaction. The disclosed methods may be useful in identifying or detecting genetic abnormalities from a subject, for example, fetal aneuploidy (e.g., trisomy 21, trisomy 18, etc.).

In some aspects, the present disclosure provides dPCR methods of multiplex quantitation for non-invasive prenatal diagnosis of aneuploidies. As cell-free fetal DNA (cffDNA) accounts for only a small percentage of the total cell-free DNA in maternal plasma, partitioning a sample into approximately 20,000 evenly sized individual partitions—each about one nanoliter in volume—may be used for absolute quantification with high specificity and sensitivity without the need to classify or quantify the concentration of target sequences present in individual partitions in the reaction. Methods for quantification are described in more detail elsewhere herein, and include, for example, peak minimum thresholding, midpoint thresholding, partial probability summation, and direct probability summation. In one example, multiple nucleic acid sequences (e.g., loci) corresponding with chromosome 21 are amplified, and the set of signals generated by all the sequences are detected in a single color channel, without ever determining the quantity of any of the individual sequences in any individual partition. In parallel, multiple nucleic acid sequences (e.g., loci) corresponding with chromosome 18 are amplified, and the set of signals generated by all the sequences are detected in a single color channel, without ever determining the quantity of any of the individual sequences in any individual partition. These two sets of signals are then compared. In some cases, a ratio of chromosome 21 to chromosome 18 is determined. Determining a ratio may be useful in identifying an increase or decrease in an amount of a chromosome in a sample (e.g., cffDNA) relative to a reference value (e.g., maternal DNA).

In an example embodiment, multiple sequences (e.g., loci) corresponding with chromosome 21 are amplified and a first sum signal generated by all sequences is determined, without ever determining the quantity of any of the individual sequences. In parallel, multiple sequences (e.g., loci) corresponding with chromosome 18 are amplified and a second sum signal generated by all sequences is determined, without ever determining the quantity of any of the individual sequences. These first and second sum signals are then compared, and a ratio is determined. Therefore, if the sum signal from four chromosome 21 sequences is determined to be 500 units, and the sum signal from four chromosome 18 sequences is determined to be 300 units, then a chromosome 21:chromosome 18 ratio of 1.6 is determined.

### Quantifying Target Nucleic Acid

Described herein, in some aspects, is a method of quantifying a nucleic acid target in a sample. First, a mixture may be provided comprising a plurality of nucleic acid molecules and a plurality of oligonucleotide probes. The plurality of nucleic acid molecules may be derived from, and/or may correspond with, the nucleic acid target in the sample. The plurality of oligonucleotide probes may each correspond to a different region of the nucleic acid target. The mixture may further comprise other reagents (e.g., amplification reagents) including, for example, oligonucleotide primers, dNTPs, a nucleic acid enzyme (e.g., a polymerase), and salts (e.g., Ca²⁺, Mg²⁺, etc.). Next, the mixture may be partitioned into a plurality of partitions (e.g., wells, microwells, droplets, etc.). Next, a plurality of signals may be generated in the plurality of partitions. The plurality of signals may be detectable in one color channel. The plurality of signals may be detectable in multiple color channels. At least one signal of the plurality of signals may correspond with the presence of a unique combination of two or more of the plurality of nucleic acid molecules in a single partition. For example, one signal may correspond to the presence of two nucleic acid molecules (e.g., two copies of a nucleic acid sequence) in a single droplet. Next, the plurality of signals may be detected in multiple partitions of the plurality of partitions. The plurality of signals may be detected in a single color channel. The plurality of signals may be detected in multiple color channels. Based on the detecting, the nucleic acid target in the sample may be quantified.

In some cases, the sample further comprises an additional plurality of nucleic acid molecules and an additional plurality of oligonucleotide probes. The additional plurality of nucleic acid molecules may be derived from and/or correspond with an additional nucleic acid target. The additional plurality of oligonucleotide probes may each correspond to a different region of the additional nucleic acid target.

A sample may be a biological sample. A sample may be derived from a biological sample. A biological sample may be, for example, blood, plasma, serum, urine, saliva, mucosal excretions, sputum, stool or tears. A biological sample may be a fluid sample. A fluid sample may be blood or plasma. A biological sample may comprise cell-free nucleic acid (e.g., cell-free RNA, cell-free DNA, etc.).

A nucleic acid target may be a nucleic acid from a pathogen (e.g., virus, bacteria, etc.). A nucleic acid target may be a nucleic acid (e.g., a chromosome) suspected of comprising one or more mutations. A nucleic acid target may be a cancer nucleic acid. A nucleic acid target may be a chromosome. Oligonucleotide probes may correspond to different regions (e.g., loci) of a chromosome. A chromosome may be chromosome 21, chromosome 18, chromosome 15, chromosome 13, an X chromosome, or a Y chromosome.

The plurality of signals may be generated by one or more of the plurality of probes from the mixture. The plurality of signals may be generated by nucleic acid amplification (e.g., PCR) of the plurality of nucleic acid molecules. Nucleic acid amplification may degrade the plurality of oligonucleotide probes (e.g., by activity of a nucleic acid enzyme), thereby generating the plurality of signals. A plurality of signals may be a plurality of fluorescent signals, a plurality of chemiluminescent signals, or a combination thereof.

A signal of the plurality of signals may correspond with two or more unique combinations of the plurality of nucleic acid molecules in a single partition (e.g., may be an ambiguous signal). For example, a signal may correspond with the presence of one nucleic acid molecule and may also correspond with the presence of two nucleic acid molecules.

In some cases, the method does not include a step of determining a number of nucleic acid molecules in any individual member of the plurality of partitions. Quantifying the nucleic acid target in the sample may comprise accounting for an ambiguous signal. Quantifying may comprise use of a peak minimum thresholding model. Quantifying may comprise use of a midpoint thresholding model. Quantifying may comprise use of a partial probability summation model. Quantifying may comprise use of a direct probability summation model.

### Quantifying Multiple Target Nucleic Acids

Described herein, in some aspects, is a method of quantifying multiple nucleic acid targets in a sample. First, a mixture may be provided comprising a first plurality of nucleic acid molecules, a second plurality of nucleic acid molecules, a first plurality of oligonucleotide probes, and a second plurality of oligonucleotide probes. The first plurality of nucleic acid molecules may be derived from, and/or may correspond with, the first nucleic acid target in the sample. The second plurality of nucleic acid molecules may be derived from, and/or may correspond with, the second nucleic acid target in the sample. The first plurality of oligonucleotide probes may each correspond to a different region of the first nucleic acid target. The second plurality of oligonucleotide probes may each correspond to a different region of the second nucleic acid target. The mixture may further comprise other reagents (e.g., amplification reagents) including, for example, oligonucleotide primers, dNTPs, a nucleic acid enzyme (e.g., a polymerase), and salts (e.g., Ca²⁺, Mg²⁺, etc.). Next, the mixture may be partitioned into a plurality of partitions (e.g., wells, microwells, droplets, etc.). Next, a plurality of signals may be generated in the plurality of partitions. The plurality of signals may be detectable in one color channel. The plurality of signals may be detectable in multiple color channels. Next, the plurality of signals may be detected in multiple partitions of the plurality of partitions. The plurality of signals may be detected in a single color channel. The plurality of signals may be detected in multiple color channels. Based on the detecting, the first nucleic acid target and the second nucleic acid target in the sample may be quantified.

The multiple nucleic acid targets in a sample may comprise a first plurality of nucleic acid molecules, a second plurality of nucleic acid molecules, a third plurality of nucleic acid molecules, a fourth plurality of nucleic acid molecules, a fifth plurality of nucleic acid molecules, a sixth plurality of nucleic acid molecules, a seventh plurality of nucleic acid molecules, an eighth plurality of nucleic acid molecules, and/or a ninth plurality of nucleic acid molecules. Each of the pluralities of nucleic acid molecules may separately be derived from, and/or may correspond with, a nucleic acid target in the sample.

The mixture may comprise one or more pluralities of oligonucleotide primers. The oligonucleotide primers in the mixture may comprise a first plurality of oligonucleotide primers, a second plurality of oligonucleotide primers, a third plurality of oligonucleotide primers, a fourth plurality of oligonucleotide primers, a fifth plurality of oligonucleotide primers, a sixth plurality of oligonucleotide primers, a seventh plurality of oligonucleotide primers, an eighth plurality of oligonucleotide primers, and/or a ninth plurality of oligonucleotide primers. Each of the pluralities of oligonucleotide primers may be a pair of oligonucleotide primers. Paired oligonucleotide primers may comprise a forward oligonucleotide primer and a reverse oligonucleotide primer. Each of the pluralities of oligonucleotide primers may be able to hybridize to different nucleic acids sequence. Each nucleic acid sequence may correspond to a different nucleic acid target.

The plurality of signals may be generated by one or more of the first plurality of probes and/or the second plurality of probes from the mixture. The plurality of signals may be generated by nucleic acid amplification (e.g., PCR) of the first plurality of nucleic acid molecules and/or the second plurality of nucleic acid molecules. Nucleic acid amplification may degrade the first and second pluralities of oligonucleotide probes (e.g., by activity of a nucleic acid enzyme), thereby generating the plurality of signals. A plurality of signals may be a plurality of fluorescent signals, a plurality of chemiluminescent signals, or a combination thereof.

The plurality of signals may be generated by one or more of a plurality of probes. The plurality of signals may be generated by a first plurality of probes, a second plurality of probes, a third plurality of probes, a fourth plurality of probes, a fifth plurality of probes, a sixth plurality of probes, a seventh plurality of probes, a eighth plurality of probes, and/or a ninth plurality of probes. The plurality of signals may be generated by nucleic acid amplification (e.g., PCR) of any one of the pluralities of nucleic acid molecules. Nucleic acid amplification may degrade any one of the pluralities of oligonucleotide probes (e.g., by activity of a nucleic acid enzyme), thereby generating the plurality of signals. A plurality of signals may be a plurality of fluorescent signals, a plurality of chemiluminescent signals, or a combination thereof.

Quantifying the first and second nucleic acid targets may comprise determining a ratio of the first nucleic acid target to the second nucleic acid target in the sample (e.g., the quantity of the first nucleic acid target relative to the quantity of the second nucleic acid target in the sample). Quantifying the first and second nucleic acid targets may comprise determining an absolute quantity of the first and second nucleic acid targets in the sample. Quantifying the first and second nucleic acid targets may comprise determining a relative quantity of the first and second nucleic acid targets in the sample.

A sample may be a biological sample. A sample may be derived from a biological sample. A biological sample may be, for example, blood, plasma, serum, urine, saliva, mucosal excretions, sputum, stool or tears. A biological sample may be a fluid sample. A fluid sample may be blood or plasma. A biological sample may comprise cell-free nucleic acid (e.g., cell-free RNA, cell-free DNA, etc.).

A nucleic acid target may be a nucleic acid from a pathogen (e.g., virus, bacteria, etc.). A nucleic acid target may be a nucleic acid (e.g., a chromosome) suspected of comprising one or more mutations. A nucleic acid target may be a cancer nucleic acid. A nucleic acid target may be a chromosome. Oligonucleotide probes may correspond to different regions (e.g., loci) of a chromosome. A chromosome may be chromosome 21, chromosome 18, chromosome 15, chromosome 13, an X chromosome, or a Y chromosome.

At least one signal of the plurality of signals may correspond with the presence of a unique combination of two or more of the first or second pluralities of nucleic acid molecules in a single partition. For example, one signal may correspond to the presence of two nucleic acid molecules (e.g., two copies of a nucleic acid sequence) in a single droplet. A signal of the plurality of signals may correspond with two or more unique combinations of the first or second pluralities of nucleic acid molecules in a single partition (e.g., may be an ambiguous signal). For example, a signal may correspond with the presence of one nucleic acid molecule and may also correspond with the presence of two nucleic acid molecules.

In some cases, the method does not include a step of determining a number of nucleic acid molecules in any individual member of the plurality of partitions. Quantifying the nucleic acid target in the sample may comprise accounting for an ambiguous signal. Quantifying may comprise use of a peak minimum thresholding model. Quantifying may comprise use of a midpoint thresholding model. Quantifying may comprise use of a partial probability summation model. Quantifying may comprise use of a direct probability summation model.

### Quantifying Target Nucleic Acid Molecules Using Probability Determination

Described herein, in some aspects, is a method of quantifying target nucleic acid molecules in a sample. First a plurality of nucleic acid molecules may be partitioned into a plurality of partitions (e.g., microwells, wells, droplets, etc.). The plurality of nucleic acid molecules may be derived from, and/or may correspond with, the target nucleic acid molecules in the sample. In addition to the plurality of nucleic acid molecules, other reagents (e.g., amplification reagents) may be partitioned, including, for example, oligonucleotide primers, oligonucleotide probes, dNTPs, a nucleic acid enzyme (e.g., a polymerase), and salts (e.g., Ca²⁺, Mg²⁺, etc.). Next, the plurality of nucleic acid molecules may be amplified, thereby generating a plurality of signals. The plurality of signals may be detectable in one color channel. The plurality of signals may be detectable in multiple color channels. Next, the plurality of signals may be detected. The plurality of signals may be detected in a single color channel. The plurality of signals may be detected in multiple color channels. Next, for each partition of the plurality of partitions, a probability that one or more nucleic acid molecule(s) of the plurality of nucleic acid molecules is present may be determined, thereby generating a plurality of probabilities. The plurality of probabilities may be generated based on the detecting (e.g., based on analysis of detected signals). The target nucleic acid molecules in the sample may be quantified based on a function of the plurality of probabilities.

The function of the plurality of probabilities may be a sum. In this case, quantifying the target nucleic acid molecules may comprise calculating a sum of all of the plurality of probabilities. The function of the plurality of probabilities may be a function derived from the properties (e.g., size, shape, width, etc.) of the signal generated from each partition. For example, a function may be generated based on the width of each signal and used to quantify the plurality of target nucleic acid molecules.

The method may further comprise partitioning, into the plurality of partitions, a plurality of oligonucleotide probes corresponding to the plurality of nucleic acid molecules. The plurality of signals may be generated from the plurality of oligonucleotide probes. The plurality of signals may be generated by nucleic acid amplification (e.g., PCR) of the plurality of nucleic acid molecules. Nucleic acid amplification may degrade the plurality of oligonucleotide probes (e.g., by activity of a nucleic acid enzyme), thereby generating the plurality of signals. Nucleic acid amplification may release a signal tag from the plurality of probes, thereby generating the plurality of signals. A plurality of signals may be a plurality of fluorescent signals, a plurality of chemiluminescent signals, or a combination thereof.

A sample may be a biological sample. A sample may be derived from a biological sample. A biological sample may be, for example, blood, plasma, serum, urine, saliva, mucosal excretions, sputum, stool or tears. A biological sample may be a fluid sample. A fluid sample may be blood or plasma. A biological sample may comprise cell-free nucleic acid (e.g., cell-free RNA, cell-free DNA, etc.).

A nucleic acid target may be a nucleic acid from a pathogen (e.g., virus, bacteria, etc.). A nucleic acid target may be a nucleic acid (e.g., a chromosome) suspected of comprising one or more mutations. A nucleic acid target may be a cancer nucleic acid. A nucleic acid target may be a chromosome. Oligonucleotide probes may correspond to different regions (e.g., loci) of a chromosome. A chromosome may be chromosome 21, chromosome 18, chromosome 15, chromosome 13, an X chromosome, or a Y chromosome.

At least one signal of the plurality of signals may correspond with the presence of a unique combination of two or more of the plurality of nucleic acid molecules in a single partition. For example, one signal may correspond to the presence of two nucleic acid molecules (e.g., two copies of a nucleic acid sequence) in a single droplet. A signal of the plurality of signals may correspond with two or more unique combinations of the plurality of nucleic acid molecules in a single partition (e.g., may be an ambiguous signal). For example, a signal may correspond with the presence of one nucleic acid molecule and may also correspond with the presence of two nucleic acid molecules.

Quantifying the target nucleic acid molecules may comprise determining a ratio of a first nucleic acid target to a second nucleic acid target in the sample (e.g., the quantity of the first nucleic acid target relative to the quantity of the second nucleic acid target in the sample). Quantifying the target nucleic acid molecules may comprise determining an absolute quantity of the target nucleic acid molecules in the sample. Quantifying the first and second nucleic acid targets may comprise determining a relative quantity of target nucleic acid molecules in the sample.

The method may not include a step of determining a number of nucleic acid molecules in any individual member of the plurality of partitions. The method may not include a step of determining a number of partitions comprising a nucleic acid sequence corresponding to the target nucleic acid molecules. The method may not include any step of determining a quantity from any individual partition of the plurality of partitions. Quantifying the target nucleic acid molecules in the sample may comprise accounting for an ambiguous signal. Quantifying may comprise use of a peak minimum thresholding model. Quantifying may comprise use of a midpoint thresholding model. Quantifying may comprise use of a partial probability summation model. Quantifying may comprise use of a direct probability summation model.

### Quantifying Target Nucleic Acid Molecules Using Direct Analysis

Described herein, in some aspects, is a method of quantifying target nucleic acid molecules in a sample. First a plurality of nucleic acid molecules may be partitioned into a plurality of partitions (e.g., microwells, wells, droplets, etc.). The plurality of nucleic acid molecules may be derived from, and/or may correspond with, the target nucleic acid molecules in the sample. In addition to the plurality of nucleic acid molecules, other reagents (e.g., amplification reagents) may be partitioned, including, for example, oligonucleotide primers, oligonucleotide probes, dNTPs, a nucleic acid enzyme (e.g., a polymerase), and salts (e.g., Ca²⁺, Mg²⁺, etc.). Next, the plurality of nucleic acid molecules may be amplified, thereby generating a plurality of signals. The plurality of signals may be detectable in one color channel. The plurality of signals may be detectable in multiple color channels. Next, the plurality of signals may be detected. The plurality of signals may be detected in a single color channel. The plurality of signals may be detected in multiple color channels. Next, the members of the plurality of signals may be compared to one another. The target nucleic acid molecules in the sample may be quantified based on the comparing. The method may not include a step of quantifying the plurality of nucleic acid molecules in any individual member of the plurality of partitions.

Comparing members of a plurality of signals to one another may comprise generating one or more signal distribution curves from the plurality of signals and analyzing the one or more signal distribution curves. Comparing may comprise measuring an area under the curve (AUC) for one or more signal distribution curves generated from the plurality of signals. The comparing may comprise comparing an AUC to a reference value. The comparing may comprise generating one or more signal distribution curves and comparing an AUC for each of the one or more signal distribution curves to one another.

The method may further comprise partitioning, into the plurality of partitions, a plurality of oligonucleotide probes corresponding to the plurality of nucleic acid molecules. The plurality of signals may be generated from the plurality of oligonucleotide probes. The plurality of signals may be generated by nucleic acid amplification (e.g., PCR) of the plurality of nucleic acid molecules. Nucleic acid amplification may degrade the plurality of oligonucleotide probes (e.g., by activity of a nucleic acid enzyme), thereby generating the plurality of signals. Nucleic acid amplification may release a signal tag from the plurality of probes, thereby generating the plurality of signals. A plurality of signals may be a plurality of fluorescent signals, a plurality of chemiluminescent signals, or a combination thereof.

A sample may be a biological sample. A sample may be derived from a biological sample. A biological sample may be, for example, blood, plasma, serum, urine, saliva, mucosal excretions, sputum, stool or tears. A biological sample may be a fluid sample. A fluid sample may be blood or plasma. A biological sample may comprise cell-free nucleic acid (e.g., cell-free RNA, cell-free DNA, etc.).

A nucleic acid target may be a nucleic acid from a pathogen (e.g., virus, bacteria, etc.). A nucleic acid target may be a nucleic acid (e.g., a chromosome) suspected of comprising one or more mutations. A nucleic acid target may be a cancer nucleic acid. A nucleic acid target may be a chromosome. Oligonucleotide probes may correspond to different regions (e.g., loci) of a chromosome. A chromosome may be chromosome 21, chromosome 18, chromosome 15, chromosome 13, an X chromosome, or a Y chromosome.

At least one signal of the plurality of signals may correspond with the presence of a unique combination of two or more of the plurality of nucleic acid molecules in a single partition. For example, one signal may correspond to the presence of two nucleic acid molecules (e.g., two copies of a nucleic acid sequence) in a single droplet. A signal of the plurality of signals may correspond with two or more unique combinations of the plurality of nucleic acid molecules in a single partition (e.g., may be an ambiguous signal). For example, a signal may correspond with the presence of one nucleic acid molecule and may also correspond with the presence of two nucleic acid molecules.

Quantifying the target nucleic acid molecules may comprise determining a ratio of a first nucleic acid target to a second nucleic acid target in the sample (e.g., the quantity of the first nucleic acid target relative to the quantity of the second nucleic acid target in the sample). Quantifying the target nucleic acid molecules may comprise determining an absolute quantity of the target nucleic acid molecules in the sample. Quantifying the first and second nucleic acid targets may comprise determining a relative quantity of target nucleic acid molecules in the sample.

The method may not include a step of determining a number of nucleic acid molecules in any individual member of the plurality of partitions. The method may not include a step of determining a number of partitions comprising a nucleic acid sequence corresponding to the target nucleic acid molecules. The method may not include any step of determining a quantity from any individual partition of the plurality of partitions. Quantifying the target nucleic acid molecules in the sample may comprise accounting for an ambiguous signal. Quantifying may comprise use of a peak minimum thresholding model. Quantifying may comprise use of a midpoint thresholding model. Quantifying may comprise use of a partial probability summation model. Quantifying may comprise use of a direct probability summation model.

### Determining a Ratio

Described herein, in some aspects, is a method of determining a quantify of a first target nucleic acid relative to a quantity of a second target nucleic acid in a sample. First, a mixture may be provided comprising a first plurality of nucleic acid molecules and a second plurality of nucleic acid molecules. The first plurality of nucleic acid molecules may be derived from, and/or may correspond with, the first nucleic acid target in the sample. The second plurality of nucleic acid molecules may be derived from, and/or may correspond with, the second nucleic acid target in the sample. In addition to the first and second pluralities of nucleic acid molecules, other reagents (e.g., amplification reagents) may be provided in the mixture, including, for example, oligonucleotide primers, oligonucleotide probes, dNTPs, a nucleic acid enzyme (e.g., a polymerase), and salts (e.g., Ca²⁺, Mg²⁺, etc.). Next, the mixture may be partitioned into a plurality of partitions (e.g., microwells, wells, droplets, etc.). Next, the first plurality of nucleic acid molecules and the second plurality of nucleic acid molecules may be amplified, thereby generating a plurality of signals. The plurality of signals may be detectable in one color channel. The plurality of signals may be detectable in multiple color channels. Next, the plurality of signals may be detected. The plurality of signals may be detected in a single color channel. The plurality of signals may be detected in multiple color channels. Next, based on the detecting, a ratio may be determined which is representative of a quantity of the first target nucleic acid relative to a quantity of the second target nucleic acid in the sample. The method may not include a step of quantifying, in any individual member of said plurality of partitions, the first plurality of nucleic acid molecules or the second plurality of nucleic acid molecules. The first target nucleic acid and the second target nucleic acid in the sample may be quantified based on the ratio.

The method may further comprise partitioning, into the plurality of partitions, a plurality of oligonucleotide probes corresponding to the plurality of nucleic acid molecules. The plurality of signals may be generated from the plurality of oligonucleotide probes. The plurality of signals may be generated by nucleic acid amplification (e.g., PCR) of the plurality of nucleic acid molecules. Nucleic acid amplification may degrade the plurality of oligonucleotide probes (e.g., by activity of a nucleic acid enzyme), thereby generating the plurality of signals. Nucleic acid amplification may release a signal tag from the plurality of probes, thereby generating the plurality of signals. A plurality of signals may be a plurality of fluorescent signals, a plurality of chemiluminescent signals, or a combination thereof.

A ratio may be representative of a fetal fraction. A ratio may be a fetal fraction. A ratio may be representative or indicative of a chromosomal abnormality. A chromosomal abnormality may be an aneuploidy. An aneuploidy may be a fetal aneuploidy. A chromosomal abnormality may be a mutation (e.g., insertion, deletion, point mutation, translocation, amplification, etc.).

A sample may be a biological sample. A sample may be derived from a biological sample. A biological sample may be, for example, blood, plasma, serum, urine, saliva, mucosal excretions, sputum, stool or tears. A biological sample may be a fluid sample. A fluid sample may be blood or plasma. A biological sample may comprise cell-free nucleic acid (e.g., cell-free RNA, cell-free DNA, etc.).

A nucleic acid target may be a nucleic acid from a pathogen (e.g., virus, bacteria, etc.). A nucleic acid target may be a nucleic acid (e.g., a chromosome) suspected of comprising one or more mutations. A nucleic acid target may be a cancer nucleic acid. A nucleic acid target may be a chromosome. Oligonucleotide probes may correspond to different regions (e.g., loci) of a chromosome. A chromosome may be chromosome 21, chromosome 18, chromosome 15, chromosome 13, an X chromosome, or a Y chromosome.

At least one signal of the plurality of signals may correspond with the presence of a unique combination of two or more of the first or second pluralities of nucleic acid molecules in a single partition. For example, one signal may correspond to the presence of two nucleic acid molecules (e.g., two copies of a nucleic acid sequence) in a single droplet. A signal of the plurality of signals may correspond with two or more unique combinations of the first or second pluralities of nucleic acid molecules in a single partition (e.g., may be an ambiguous signal). For example, a signal may correspond with the presence of one nucleic acid molecule and may also correspond with the presence of two nucleic acid molecules.

Determining the ratio may comprise generating a signal map from the plurality of signals. A signal map may be a graph or other display representative of properties of a signal (e.g., wavelength, amplitude, etc.). A signal map may comprise a plurality of target populations. A signal map may comprise an overlapping region between each of the plurality of target populations. At least a portion of the plurality of target populations overlap with one another.

In some cases, the method does not include a step of determining a number of nucleic acid molecules in any individual member of the plurality of partitions. Quantifying the first and second nucleic acid target in the sample may comprise accounting for an ambiguous signal. Quantifying may comprise use of a peak minimum thresholding model. Quantifying may comprise use of a midpoint thresholding model. Quantifying may comprise use of a partial probability summation model. Quantifying may comprise use of a direct probability summation model.

The first plurality of nucleic acid molecules may be copies of the first nucleic acid target, where the copies have been transferred from the sample into the mixture. The second plurality of nucleic acid molecules may be copies of the second nucleic acid target, where the copies have been transferred from the sample into the mixture. The first plurality of nucleic acid molecules and the second plurality of nucleic acid molecules may originate from the sample. The first plurality of nucleic acid molecules may be products of nucleic acid amplification (e.g., PCR) of the first target nucleic acid. The second plurality of nucleic acid molecules may be products of nucleic acid amplification (e.g., PCR) of the second target nucleic acid. The first plurality of nucleic acid molecules may be products of nucleic acid extension of the first target nucleic acid. The second plurality of nucleic acid molecules may be products of nucleic acid extension of the second target nucleic acid. The first plurality of nucleic acid molecules may be products of reverse transcription of the first target nucleic acid. The second plurality of nucleic acid molecules may be products of nucleic acid extension of the second target nucleic acid.

### Multiplex Calibration

When multiplexing, dPCR methods may involve creating spatially resolved clusters of fluorescent points and drawing a “cutoff” or “threshold” between the clusters to identify whether an individual droplet is a “positive” cluster, containing the target of interest, or a “negative” cluster, not containing the target of interest. Thus, only after “gating” droplets into groups can the exact number of template-positive droplets be measured. However, gating may suffer from inaccuracy due to high levels of subjectivity. Therefore calibrating the location of multiple clusters in a multiplex assay normalizes the locations of the target clusters in an assay, thus mitigating inaccuracies. When multiplexing multiple targets into two channels of a digital PCR reaction, there may be overlap between many of the final states. Accordingly, synthetic or natural targets may be used in a formulation for digital PCR in order to calibrate the location of multiple clusters in a multiplexed approach.

The addition of a template and one or more synthetic (or otherwise generated to be different from the target of interest) primer/probe sets into the mix for the digital PCR reaction (at a known signal ratio to the other targets in the multiplex reaction) ensures that some, but not most, of the droplets generate a “positive” fluorescence. The resulting distinct cluster can be used to normalize the locations of the target clusters in the assay. Additionally or alternatively to addition of template nucleic acid, calibration methods may include spiking known samples into a reaction, using positive controls expected to be present, or, in the case of digital droplet PCR, generating known positive droplets in another reaction and adding them to the reaction vessel before sensing. These clusters could be distinct from the other targets in the assay or in the same locations.

### Digital Assays

In some aspects, the present disclosure provides assays for unambiguously detecting the presence or absence of multiple nucleic acid targets in a sample. Nucleic acid target detection may be accomplished by the use of two or more reactions. For example, an assay for measuring a plurality of nucleic acid targets may comprise a first reaction and a second reaction. Both a first and second reaction may, alone, fail to non-degenerately detect the presence or absence of any combination of nucleic acid targets. The results of the first and second reactions may together unambiguously detect the presence or absence of each of the nucleic acid targets.

Any number of nucleic acid targets may be detected using assays of the present disclosure. In some cases, an assay may unambiguously detect at least 3, 4, 5, 6, 7, 8, 9, 10, 11, 12, 13, 14, 15, 20, 30, 40, 50 nucleic acid targets, or more. In some cases, an assay may unambiguously detect at most 50, 40, 30, 20, 15, 14, 13, 12, 11, 10, 9, 8, 7, 6, 5, 4, or 3 nucleic acid targets. An assay may comprise any number of reactions, where the results of the reactions together identify a plurality of nucleic acid targets, in any combination of presence or absence. An assay may comprise 2, 3, 4, 5, 6, 7, 8, 9, 10 reactions, or more. Each reaction may be individually incapable of non-degenerately detecting the presence or absence of any combination of nucleic acid targets. However, the results of each reaction together may unambiguously detect the presence or absence of each of the nucleic acid targets.

Reactions may be performed in the same sample solution volume. For example, a first reaction may generate a fluorescent signal in a first color channel, while a second reaction may generate a fluorescent signal in a second color channel, thereby generating two measurements for comparison. Alternatively, reactions may be performed in different sample solution volumes. For example, a first reaction may be performed in a first sample solution volume and generate a fluorescent signal in a given color channel, and a second reaction may be performed in a second sample solution volume and generate a fluorescent signal in the same color channel or a different color channel, thereby generating two measurements for comparison

In some aspects, the present disclosure provides methods for performing a digital assay. A method for performing a digital assay may comprise partitioning a plurality of nucleic acid targets and a plurality of oligonucleotide probes into a plurality of partitions. In some cases, two, three, four, five, or more nucleic acid targets may be partitioned into a plurality of partitions together with two, three, four, five, six, seven, eight, nine, ten, 11, 12, 13, 14, 15, 16, 17, 18, 19, 20 or more oligonucleotide probes. Following partitioning, the nucleic acid targets may be amplified in the partitions, for example, by polymerase chain reaction (PCR). Next, N signals may be generated from the oligonucleotide probes. Each signal of the N signals may correspond to the presence of a unique combination of nucleic acid targets in a partition. Following signal generation, the N signals may be detected in a single optical channel. The signals may be detected using, for example, fluorescence detection in a single-color channel.

A method for performing a digital assay may comprise amplifying nucleic acid targets derived from a sample in a plurality of partitions comprising oligonucleotide probes complementary to one or more regions of nucleic acid targets. Each oligonucleotide probe may be labeled with a fluorophore. The fluorophores may be capable of being detected in a single optical channel. For example, the fluorophores may each comprise similar emission wavelength spectra, such that they can be detected in a single optical channel. Following partitioning, N signals may be detected from the plurality of partitions if one or more of the nucleic acid targets is present. Each of the N signals may correspond to a unique combination of one or more of the nucleic acid targets present in a partition. From the N signals, the presence or absence of each of the nucleic acid targets in the sample may be determined.

A reaction may comprise generating a cumulative signal measurement. Assays of the present disclosure may comprise comparing two or more cumulative signal measurements to unambiguously detect any combination of nucleic acid targets in a sample. A cumulative signal measurement may comprise one or more signals generated from one or more probes provided to a sample solution. A cumulative signal measurement may be a signal intensity level which corresponds to the sum of signals generated from multiple oligonucleotide probes. For example, two probes may each bind to a nucleic acid molecule, where each probe generates a signal of a given wavelength at 1× intensity. Measurement of these signals would generate a cumulative signal measurement corresponding to the sum of both signal intensities, namely a 2× signal intensity.

A reaction may comprise an ambiguity. An ambiguity may be a signal that fails to unambiguously identify a single combination of nucleic acid targets in a sample. For example, a reaction may generate a signal at 2× intensity level. Based on the encoding of the reaction (e.g., the concentration of hybridization probes present in the reaction), a 2× intensity level may correspond to more than one combination of nucleic acid targets, thereby comprising an ambiguity. An ambiguity may be resolved by performing one or more additional reactions, thereby resolving the ambiguity. For example, a second reaction may generate a 3× signal intensity level, where the presence of both a 2× signal intensity level from a first reaction and a 3× signal intensity level from a second reaction uniquely identifies a given combination of nucleic acid targets from a sample.

An assay may comprise selecting two or more reactions from a selection of reactions, depending on the information necessary to resolve an ambiguity. For example, a first reaction may comprise an ambiguity at a first signal level and a second signal level. Results corresponding to the first signal level may identify a first additional reaction as necessary to resolve the ambiguity, while results corresponding to the second signal level may identify a second additional reaction as necessary to resolve the ambiguity.

### Amplification

In some aspects, the disclosed methods comprise nucleic acid amplification. Amplification conditions may comprise thermal cycling conditions, including temperature and length in time of each thermal cycle. The use of particular amplification conditions may serve to modify the signal intensity of a signal, thereby enabling a signal (or plurality of signals) to correspond to a unique combination of nucleic acid targets. Amplification may comprise using enzymes such to produce additional copies of a nucleic. The amplification reaction may comprise using oligonucleotide primers as described elsewhere herein. The oligonucleotide primers may use specific sequences to amplify a specific sequence. The oligonucleotide primers may amplify a specific sequence by hybridizing to a sequence upstream and downstream of the primers and result in amplifying the sequence inclusively between the upstream and downstream primer. The oligonucleotide may be able to amplify more than one sequence analyte by hybridizing upstream or downstream of multiple different sequences. The amplification reaction may comprise the use of nucleotide tri-phosphate reagents. The nucleotide tri-phosphate reagents may comprise using deoxyribo-nucleotide tri-phosphate (dNTPs). The nucleotide tri-phosphate reagents may be used as precursors to the amplified nucleic acids. The amplification reaction may comprise using oligonucleotide probes as described elsewhere herein. The amplification reaction may comprise using enzymes. Non-limiting examples of enzymes include thermostable enzymes, DNA polymerases, RNA polymerases, and reverse transcriptases. The amplification reaction may comprise generating nucleic acid molecules of a different nucleotide types. For example, a target nucleic acid may comprise DNA and an RNA molecule may be generated. In another example, an RNA molecule may be subjected to an amplification reaction and a cDNA molecule may be generated.

The amplification may be performed in an isothermal process. “Isothermal” means conducting a reaction at a substantially constant temperature, i.e., without varying the reaction temperature in which a nucleic acid polymerization reaction occurs. Isothermal temperatures for isothermal amplification reactions are generally below the melting temperature (Tm; the temperature at which half of the potentially double-stranded molecules in a mixture are in a single-stranded, denatured state) of the predominant reaction product, i.e., generally 90° C. or below, usually between about 50° C. and 75° C., and preferably between about 55° C. to 70° C., or 60° C. to 70° C., or more preferably at about 65° C. Although the polymerization reaction may occur in isothermal conditions, an isothermal process may optionally include a pre-amplification heat denaturation step to generate a single-stranded target nucleic acid to be used in the isothermal amplifying step.

The isothermal amplification may be linear isothermal amplification or exponential isothermal amplification. Isothermal linear amplification processes amplify template nucleic acid and not amplification products under isothermal conditions, may be conducted using only one amplification primer, and generally amplify a target sequence by about 1,000 fold within one hour. Isothermal exponential amplification processes use a product of an amplification reaction as a substrate in a subsequent step in the amplification reaction that uses isothermal conditions to amplify a target sequence about 10,000-fold to 100,000-fold within one hour. “Amplification conditions” refer to the cumulative biochemical and physical conditions in which an amplification reaction is conducted, which may be designed based on well-known standard methods.

### Partitioning

Methods of the present disclosure may comprise partitioning a sample or mixture into a plurality of partitions. A sample of mixture may comprise nucleic acids, oligonucleotide probes, and/or additional reagents into a plurality of partitions. A partition may be a droplet (e.g., a droplet in an emulsion). A partition may be a microdroplet. A partition may be a well. A partition may be a microwell. Partitioning may be performed using a microfluidic device. In some cases, partitioning is performed using a droplet generator. Partitioning may comprise dividing a sample or mixture into water-in-oil droplets. A droplet may comprise one or more nucleic acids. A droplet may comprise a single nucleic acid. A droplet may comprise two or more nucleic acids. A droplet may comprise no nucleic acids. Each droplet of a plurality of droplets may generate a signal. A plurality of signals may comprise the signal(s) generated from each of a plurality of droplets comprising a subset of a sample.

The plurality of partitions may be at least 10, 20, 30, 40, 50, 60, 70, 80, 90, 100, 200, 300, 400, 500, 600, 700, 800, 900, 1,000, 2,000, 3,000, 4,000, 5,000, 6,000, 7,000, 8,000, 9,000, 10,000, 11,000, 12000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 40,000, 50,000, 60,000, 70,000, 80,000, 90,000, 100,000 or more partitions. The plurality of partitions may be no more than 100,000, 90,000, 80,000, 70,000, 60,000, 50,000, 40,000, 30,000, 29,000, 28,000, 27,000, 26,000, 25,000, 24,000, 23,000, 22,000, 21,000, 20,000, 19,000, 18,000, 17,000, 16,000, 15,000, 14,000, 13,000, 12,000, 11,000, 10,000, 9,000, 8,000, 7,000, 6,000, 5,000, 4,000, 3,000, 2,000, 1,000, 900, 800, 700, 600, 500, 400, 300, 200, 100, 90, 80, 70, 60, 50, 40, 30, 20, 10, 5 or fewer. The plurality of partitions may be defined by a range of any of the foregoing values.

The methods and systems of the present disclosure allow for quantification without using partitions that comprise more than one target. It allows for quantification without using partitions that comprise more than one target if less than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% of partitions comprise more than one targets. It allows for quantification without using partitions that comprise more than one target if more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more partitions comprise more than one target.

The methods and systems of the present disclosure allows for quantification without using partitions that comprise an ambiguous signal. It allows for quantification without using partitions that comprise an ambiguous signal if less than 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% of partitions comprise an ambiguous signal. It allows for quantification without using partitions that an ambiguous signal if more than 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50% or more partitions comprise an ambiguous signal.

### Thermal Cycling

Methods of the present disclosure may comprise thermal cycling. Thermal cycling may comprise one or more thermal cycles. Thermally cycling may be performed under reaction conditions appropriate to amplify a template nucleic acid with PCR. Amplification of a template nucleic acid may require binding or annealing of oligonucleotide primer(s) to the template nucleic acid. Appropriate reaction conditions may include appropriate temperature conditions, appropriate buffer conditions, and the presence of appropriate reagents. Appropriate temperature conditions may, in some cases, be such that each thermal cycle is performed at a desired annealing temperature. A desired annealing temperature may be sufficient for annealing of an oligonucleotide probe(s) to a nucleic acid target. Appropriate buffer conditions may, in some cases, be such that the appropriate salts are present in a buffer used during thermal cycling. Appropriate salts may include magnesium salts, potassium salts, ammonium salts. Appropriate buffer conditions may be such that the appropriate salts are present in appropriate concentrations. Appropriate reagents for amplification of each member of a plurality of nucleic acid targets with PCR may include deoxytriphosphates (dNTPs). dNTPs may comprise natural or non-natural dNTPs including, for example, dATP, dCTP, dGTP, dTTP, dUTP, and variants thereof.

In various aspects, primer extension reactions are utilized to generate amplified product. Primer extension reactions generally comprise a cycle of incubating a reaction mixture at a denaturation temperature for a denaturation duration and incubating a reaction mixture at an elongation temperature for an elongation duration. In any of the various aspects, multiple cycles of a primer extension reaction can be conducted. Any suitable number of cycles may be conducted. For example, the number of cycles conducted may be less than about 100, 90, 80, 70, 60, 50, 40, 30, 20, 10, or 5 cycles. The number of cycles conducted may depend upon, for example, the number of cycles (e.g., cycle threshold value (Ct)) used to obtain a detectable amplified product (e.g., a detectable amount of amplified DNA product that is indicative of the presence of a target DNA in a nucleic acid sample). For example, the number of cycles used to obtain a detectable amplified product (e.g., a detectable amount of DNA product that is indicative of the presence of a target DNA in a nucleic acid sample) may be less than about or about 100 cycles, 75 cycles, 70 cycles, 65 cycles, 60 cycles, 55 cycles, 50 cycles, 40 cycles, 35 cycles, 30 cycles, 25 cycles, 20 cycles, 15 cycles, 10 cycles, or 5 cycles. Moreover, in some embodiments, a detectable amount of an amplifiable product (e.g., a detectable amount of DNA product that is indicative of the presence of a target DNA in a nucleic acid sample) may be obtained at a cycle threshold value (Ct) of less than 100, 75, 70, 65, 60, 55, 50, 45, 40, 35, 30, 25, 20, 15, 10, or 5.

The time for which an amplification reaction yields a detectable amount of amplified nucleic acid may vary depending upon the nucleic acid sample, the sequence of the target nucleic acid, the sequence of the primers, the particular nucleic acid amplification reactions conducted, and the particular number of cycles of the amplification, the temperature of the reaction, the pH of the reaction. For example, amplification of a target nucleic acid may yield a detectable amount of product indicative to the presence of the target nucleic acid at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

In some embodiments, amplification of a nucleic acid may yield a detectable amount of amplified DNA at time period of 120 minutes or less; 90 minutes or less; 60 minutes or less; 50 minutes or less; 45 minutes or less; 40 minutes or less; 35 minutes or less; 30 minutes or less; 25 minutes or less; 20 minutes or less; 15 minutes or less; 10 minutes or less; or 5 minutes or less.

### Nucleic Acid Targets

A nucleic acid target of the present disclosure may be derived from a biological sample. A biological sample may be a sample derived from a subject. A biological sample may comprise any number of macromolecules, for example, cellular macromolecules. A biological sample may be derived from another sample. A biological sample may be a tissue sample, such as a biopsy, core biopsy, needle aspirate, or fine needle aspirate. A biological sample may be a fluid sample, such as a blood sample, urine sample, or saliva sample. A biological sample may be a skin sample. A biological sample may be a cheek swab. A biological sample may be a plasma or serum sample. A biological sample may comprise one or more cells. A biological sample may be a cell-free sample. A cell-free sample may comprise extracellular polynucleotides. Extracellular polynucleotides may be isolated from a bodily sample that may be selected from the group consisting of blood, plasma, serum, urine, saliva, mucosal excretions, sputum, stool and tears.

A nucleic acid target may be derived from one or more cells. A cell may be a tumor cell. A cell may be a cell suspected of comprising a viral pathogen. In some cases, a nucleic acid target is derived from a cell-free sample (e.g., serum, plasma). A nucleic acid target may be cell-free nucleic acid. Cell-free nucleic acid may be, for example, cell-free tumor DNA, cell-free fetal DNA, cell-free RNA, etc. A nucleic acid target may comprise deoxyribonucleic acid (DNA). DNA may be any kind of DNA, including genomic DNA. A nucleic acid target may be viral DNA. A nucleic acid target may comprise ribonucleic acid (RNA). RNA may be any kind of RNA, including messenger RNA, transfer RNA, ribosomal RNA, and microRNA. RNA may be viral RNA. Nucleic acid targets may comprise one or more members. A member may be any region of a nucleic acid target. A member may be of any length. A member may be, for example, up to 1, 2, 3, 4, 5, 10, 20, 50, 100, 500, 1000, 5000, 10000, 50000, or 100000 nucleotides, or more. In some instances, a member may be a gene. A nucleic acid target may comprise a gene whose detection may be useful in diagnosing one or more diseases. A gene may be a viral gene or bacterial gene whose detection may be useful in identifying the presence or absence of a pathogen in a subject. In some cases, the methods of the present disclosure are useful in detecting the presence or absence or one or more infectious agents (e.g., viruses) in a subject.

In some cases, a nucleic acid target is a chromosome. One or more nucleic acid molecules analyzed by methods of the present disclosure may correspond to a chromosome. For example, nucleic acid molecules may be fragments of a chromosome. In another example, nucleic acid molecules may be amplification products of a chromosome. Nucleic acid molecules corresponding to a chromosome may be obtained from one or more cells. Nucleic acid molecules corresponding to a chromosome may be obtained from a cell-free sample (e.g., serum, plasma, blood, etc.).

### Sample Processing

A sample may be processed concurrently with, prior to, or subsequent to the methods of the present disclosure. A sample may be processed to purify or enrich for nucleic acids (e.g., to purify nucleic acids from a plasma sample). A sample comprising nucleic acids may be processed to purity or enrich for nucleic acid of interest. A sample comprising nucleic acids may be processed to enrich for fetal nucleic acid. A sample may be enriched for nucleic acid of interest (e.g., fetal nucleic acid) by various methods including, for example, sequence-specific enrichment (e.g., via use of capture sequences), epigenetic-specific enrichment (e.g., via use of methylation-specific capture moieties, such as antibodies). Enrichment may comprise isolation of nucleic acid of interest and/or depletion of nucleic acid that is not of interest. In some cases, a sample is not processed to purify or enrich for nucleic acid of interest prior to performing methods of the present disclosure (e.g., amplification of nucleic acids from a sample). For example, a sample may not be processed to enrich for fetal nucleic acid prior to mixing a sample with oligonucleotide primers and oligonucleotide probes, as described elsewhere herein.

### Nucleic Acid Enzymes

Mixtures and compositions of the present disclosure may comprise one or more nucleic acid enzymes. A nucleic acid enzyme may have exonuclease activity. A nucleic acid enzyme may have endonuclease activity. A nucleic acid enzyme may have RNase activity. A nucleic acid enzyme may be capable of degrading a nucleic acid comprising one or more ribonucleotide bases. A nucleic acid enzyme may be, for example, RNase H or RNase III. An RNase III may be, for example, Dicer. A nucleic acid may be an endonuclease I such as, for example, a T7 endonuclease I. A nucleic acid enzyme may be capable of degrading a nucleic acid comprising a non-natural nucleotide. A nucleic acid enzyme may be an endonuclease V such as, for example, an E. coli endonuclease V. A nucleic acid enzyme may be a polymerase (e.g., a DNA polymerase). A polymerase may be Taq polymerase or a variant thereof. A nucleic acid enzyme may be capable, under appropriate conditions, of degrading an oligonucleotide probe. A nucleic acid enzyme may be capable, under appropriate conditions, of releasing a quencher from an oligonucleotide probe.

### Reactions

A reaction may comprise contacting nucleic acid targets with one or more oligonucleotide probes. A reaction may comprise contacting a sample solution volume (e.g., a droplet, well, tube, etc.) with a plurality of oligonucleotide probes, each corresponding to one of a plurality of nucleic acid targets, to generate a plurality of signals generated from the plurality of oligonucleotide probes. A reaction may comprise polymerase chain reaction (PCR). A reaction may be a digital PCR reaction.

In some cases, one or more signals from a plurality of signals fail to non-degenerately identify the presence or absence of any combination of a plurality of nucleic acid molecules (e.g., a signal corresponds to two or more combinations of nucleic acid molecules in a sample volume). As disclosed herein, two or more signals may be compared, thereby non-degenerately indicating the presence or absence of a plurality of nucleic acid targets, in any combination.

In some cases, one or more synthetic (or otherwise generated to be different from the target of interest) primer/probe sets may be used to calibrate the location of multiple signals in a reaction (e.g., a digital PCR reaction). A reaction may include a known amount of template. A ratio of a known template to the other targets in a reaction may be used to normalize the locations of target clusters in an assay.

### Oligonucleotide Primers

An oligonucleotide primer (or “amplification oligomer”) of the present disclosure may be a deoxyribonucleic acid. An oligonucleotide primer may be a ribonucleic acid. An oligonucleotide primer may comprise one or more non-natural nucleotides. A non-natural nucleotide may be, for example, deoxyinosine.

An oligonucleotide primer may be a forward primer. An oligonucleotide primer may be a reverse primer. An oligonucleotide primer may be between about 5 and about 50 nucleotides in length. An oligonucleotide primer may be at least 5, 10, 11, 12, 13, 14, 15, 16, 17, 18, 19, 20, 25, 30, 35, 40, 45, or 50 base pairs in length, or more. An oligonucleotide primer may be at most 50, 45, 40, 35, 30, 25, 20, 19, 18, 17, 16, 15, 14, 13, 12, 11, 10, or 5 nucleotides in length. An oligonucleotide primer may be about 5, 10, 11, 12, 13, 14, 15, 16, 17, 18, 19, 20, 25, 30, 35, 40, 45, or 50 base pairs in length.

A set of oligonucleotide primers may comprise paired oligonucleotide primers. Paired oligonucleotide primers may comprise a forward oligonucleotide primer and a reverse oligonucleotide primer. A forward oligonucleotide primer may be configured to hybridize to a first region (e.g., a 3′ end) of a nucleic acid sequence, and a reverse oligonucleotide primer may be configured to hybridize to a second region (e.g., a 5′ end) of the nucleic acid sequence, thereby being configured to amplify the nucleic acid sequence under conditions sufficient for nucleic acid amplification. Different sets of oligonucleotide primers may be configured to amplify different nucleic acid target sequences.

A mixture may comprise a plurality of forward oligonucleotide primers. A plurality of forward oligonucleotide primers may be a deoxyribonucleic acid. Alternatively, a plurality of forward oligonucleotide primers may be a ribonucleic acid. A plurality of forward oligonucleotide primers may be between about 5 and about 50 nucleotides in length. A plurality of forward oligonucleotide primer may be at least 5, 10, 11, 12, 13, 14, 15, 16, 17, 18, 19, 20, 25, 30, 35, 40, 45, or 50 base pairs in length, or more. A plurality of forward oligonucleotide primer may be at most 50, 45, 40, 35, 30, 25, 20, 19, 18, 17, 16, 15, 14, 13, 12, 11, 10, or 5 nucleotides in length.

A mixture may comprise a plurality of reverse oligonucleotide primers. A plurality of reverse oligonucleotide primers may be a deoxyribonucleic acid. Alternatively, a plurality of reverse oligonucleotide primers may be a ribonucleic acid. A plurality of reverse oligonucleotide primers may be between about 5 and about 50 nucleotides in length. A plurality of reverse oligonucleotide primer may be at least 5, 10, 11, 12, 13, 14, 15, 16, 17, 18, 19, 20, 25, 30, 35, 40, 45, or 50 base pairs in length, or more. A plurality of reverse oligonucleotide primer may be at most 50, 45, 40, 35, 30, 25, 20, 19, 18, 17, 16, 15, 14, 13, 12, 11, 10, or 5 nucleotides in length.

In some aspects, a mixture may include one or more synthetic (or otherwise generated to be different from the target of interest) primers for digital PCR reactions. The one or more synthetic primers may be used in combination with a template to calibrate the location of multiple clusters in a reaction.

In some aspects, a mixture may be subjected to conditions sufficient to anneal an oligonucleotide primer to a nucleic acid molecule. In some aspects, a mixture may be subjected to conditions sufficient to anneal a plurality of oligonucleotide primers to a nucleic acid molecule. In some aspects, a mixture may be subjected to conditions sufficient to anneal a plurality of oligonucleotide primers to a plurality of nucleic acid targets. The mixture may be subjected to conditions which are sufficient to denature nucleic acid molecules. Subjecting a mixture to conditions sufficient to anneal an oligonucleotide primer to a nucleic acid target may comprise thermally cycling the mixture under reaction conditions appropriate to amplify the nucleic acid target(s) with, for example, polymerase chain reaction (PCR).

Conditions may be such that an oligonucleotide primer pair (e.g., forward oligonucleotide primer and reverse oligonucleotide primer) are degraded by a nucleic acid enzyme. An oligonucleotide primer pair may be degraded by the exonuclease activity of a nucleic acid enzyme. An oligonucleotide primer pair may be degraded by the RNase activity of a nucleic acid enzyme. Degradation of the oligonucleotide primer pair may result in release of the oligonucleotide primer. Once released, the oligonucleotide primer pair may bind or anneal to a template nucleic acid.

### Oligonucleotide Probes

Samples, mixtures, kits, and compositions of the present disclosure may comprise an oligonucleotide probe, also referenced herein as a “detection probe” or “probe”. An oligonucleotide probe may be a nucleic acid (e.g., DNA, RNA, etc.). An oligonucleotide probe may comprise a region complementary to a region of a nucleic acid target. The concentration of an oligonucleotide probe may be such that it is in excess relative to other components in a sample.

An oligonucleotide probe may comprise a non-target-hybridizing sequence. A non-target-hybridizing sequence may be a sequence which is not complementary to any region of a nucleic acid target sequence. An oligonucleotide probe comprising a non-target-hybridizing sequence may be a hairpin detection probe. An oligonucleotide probe comprising a non-target-hybridizing sequence may be a molecular beacon probe. Examples of molecular beacon probes are provided in, for example, U.S. Pat. No. 7,671,184, incorporated herein by reference in its entirety. An oligonucleotide probe comprising a non-target-hybridizing sequence may be a molecular torch. Examples of molecular torches are provided in, for example, U.S. Pat. No. 6,534,274, incorporated herein by reference in its entirety.

A sample may comprise more than one oligonucleotide probe. Multiple oligonucleotide probes may be the same, or may be different. An oligonucleotide probe may be at least 5, at least 10, at least 15, at least 20, or at least 30 nucleotides in length, or more. An oligonucleotide probe may be at most 30, at most 20, at most 15, at most 10 or at most 5 nucleotides in length. In some examples, a mixture comprises a first oligonucleotide probe and one or more additional oligonucleotide probes. An oligonucleotide probe may be a nucleic acid (e.g., DNA, RNA, etc.). An oligonucleotide probe may be at least 2, 3, 4, 5, 6, 7, 8, 9, 10, 20, 30, 40, or 50 nucleotides in length, or more. An oligonucleotide probe may be at most 50, 40, 30, 20, 10, 9, 8, 7, 6, 5, 4, 3, or 2 nucleotides in length.

In some cases, 2, 3, 4, 5, 6, 7, 8, 9, 10, 11, 12, 13, 14, 15, 20, or more different oligonucleotide probes are partitioned into a plurality of partitions. Each oligonucleotide probe may correspond to (e.g., capable of binding to) a given region of a nucleic acid target (e.g., a chromosome) in a sample. In one example, a first oligonucleotide probe is specific for a first region of a first nucleic acid target, a second oligonucleotide probe is specific for a second region of the first nucleic acid target, and a third oligonucleotide probe is specific for a third region of the first nucleic acid target. Each oligonucleotide probe may comprise a signal tag with about equal emission wavelengths. In some cases, each oligonucleotide probe comprises an identical fluorophore. In some cases, each oligonucleotide probe comprises a different fluorophore, where each fluorophore is capable of being detected in a single optical channel.

In some aspects, a mixture may include one or more synthetic (or otherwise generated to be different from the target of interest) probes for digital PCR reactions. The one or more synthetic probes may have a known fluorescence ratio to the other targets in the reaction and used to calibrate the location of multiple clusters in a reaction. The one or more synthetic probes may be used to normalize the locations of the target clusters in the assay by ensuring that some, but not most, of the droplets generate a “positive” fluorescence in a distinct cluster.

A probe may correspond to a region of a nucleic acid target. For example, a probe may have complementarity and/or homology to a region of a nucleic acid target. A probe may comprise a region which is complementary or homologous to a region of a nucleic acid target. A probe corresponding to a region of a nucleic acid target may be capable of binding to the region of the nucleic acid target under appropriate conditions (e.g., temperature conditions, buffer conditions, etc.). For example, a probe may be capable of binding to a region of a nucleic acid target under conditions appropriate for polymerase chain reaction. A probe may correspond to an oligonucleotide which corresponds to a nucleic acid target. For example, an oligonucleotide may be a primer with a region complementary to a nucleic acid target and a region complementary to a probe.

A probe may be a nucleic acid complementary to a region of a given nucleic acid target. Each probe used in the methods and assays of the presence disclosure may comprise at least one fluorophore. A fluorophore may be selected from any number of fluorophores. A fluorophore may be selected from three, four, five, six, seven, eight, nine, or ten fluorophores, or more. One or more oligonucleotide probes used in a single reaction may comprise the same fluorophore. In some cases, all oligonucleotide probes used in a single reaction comprise the same fluorophore. Each probe may, when excited and contacted with its corresponding nucleic acid target, generate a signal. A signal may be a fluorescent signal. A plurality of signals may be generated from one or more probes.

An oligonucleotide probe may have less than 50%, 40%, 30%, 20%, 10%, 5%, or 1% complementarity to any member of a plurality of nucleic acid targets. An oligonucleotide probe may have no complementarity to any member of the plurality of nucleic acid targets.

An oligonucleotide probe may comprise a detectable label. A detectable label may be a chemiluminescent label. A detectable label may comprise a chemiluminescent label. A detectable label may comprise a fluorescent label. A detectable label may comprise a fluorophore. A fluorophore may be, for example, FAM, TET, HEX, JOE, Cy3, or Cy5. A fluorophore may be FAM. A fluorophore may be HEX. An oligonucleotide probe may further comprise one or more quenchers. A quencher may inhibit signal generation from a fluorophore. A quencher may be, for example, TAMRA, BHQ-1, BHQ-2, or Dabcy. A quencher may be BHQ-1. A quencher may be BHQ-2.

### Signal Generation

Thermal cycling may be performed such that one or more oligonucleotide probes are degraded by a nucleic acid enzyme. An oligonucleotide probe may be degraded by the exonuclease activity of a nucleic acid enzyme. An oligonucleotide probe may generate a signal upon degradation. In some cases, an oligonucleotide probe may generate a signal only if at least one member of a plurality of nucleic acid targets is present in a mixture.

A reaction may generate one or more signals. A reaction may generate a cumulative intensity signal comprising a sum of multiple signals. A signal may be a chemiluminescent signal. A signal may be a fluorescent signal. A signal may be generated by an oligonucleotide probe. For example, excitation of a hybridization probe comprising a luminescent signal tag may generate a signal. A signal may be generated by a fluorophore. A fluorophore may generate a signal upon release from a hybridization probe. A reaction may comprise excitation of a fluorophore. A reaction may comprise signal detection. A reaction may comprise detecting emission from a fluorophore.

A signal may be a fluorescent signal. A signal may correspond to a fluorescence intensity level. Each signal measured in the methods of the present disclosure may have a distinct fluorescence intensity value, thereby corresponding to the presence of a unique combination of nucleic acid targets in a partition. A signal may be generated by one or more oligonucleotide probes. The number of signals generated in a digital assay may correspond to the number of oligonucleotide probes that are partitioned, the number of nucleic acid targets that are partitioned, and, in some cases, the partitioning conditions. For example, where three nucleic acid targets and three oligonucleotide probes are partitioned such that each partition may comprise one, two, or all three nucleic acid targets, seven signals may be generated, where each signal corresponds to the presence of a unique combination of the three nucleic acid targets in the partition. N may be a number of signals detected in a single optical channel in an assay of the present disclosure. N may be at least 2, 3, 4, 5, 6, 7, 8, 9, 10, 11, 12, 13, 14, 15, 20, 25, 30, 40, 50 or more. N may be at most 50, 40, 30, 24, 20, 15, 14, 13, 12, 11, 10, 9, 8, 7, 6, 5, 4, 3, or 2. N may be 2, 3, 4, 5, 6, 7, 8, 9, 10, 11, 12, 13, 14, 15, 20, 25, 30, 40, or 50.

As will be recognized and is described elsewhere herein, sets of signals may be generated in multiple different optical channels, where each set of signals is detected in a single optical channel, thereby significantly increasing the number of nucleic acid targets that can be measured in a single reaction (e.g., digital PCR reaction). In some cases, two sets of signals are detected in a single reaction. Each set of signals detected in a reaction may comprise the same number of signals, or different numbers of signals.

In some cases, a signal may be generated simultaneous with hybridization of an oligonucleotide probe to a region of a nucleic acid. For example, an oligonucleotide probe (e.g., a molecular beacon probe or molecular torch) may generate a signal (e.g., a fluorescent signal) following hybridization to a nucleic acid. In some cases, a signal may be generated subsequent to hybridization of an oligonucleotide probe to a region of a nucleic acid, following degradation of the oligonucleotide probe by a nucleic acid enzyme.

In cases where an oligonucleotide probe comprises a signal tag, the oligonucleotide probe may be degraded when bound to a region of an oligonucleotide primer, thereby generating a signal. For example, an oligonucleotide probe (e.g., a TaqMan® probe) may generate a signal following hybridization of the oligonucleotide probe to a nucleic acid and subsequent degradation by a polymerase (e.g., during amplification, such as PCR amplification). An oligonucleotide probe may be degraded by the exonuclease activity of a nucleic acid enzyme.

An oligonucleotide probe may comprise a quencher and a fluorophore, such that the quencher is released upon degradation of an oligonucleotide probe, thereby generating a fluorescent signal. Thermal cycling may be used to generate one or more signals. Thermal cycling may generate at least 1, 2, 3, 4, 5, 6, 7, 8, 9, 10 signals, or more. Thermal cycling may generate at most 10, 9, 8, 7, 6, 5, 4, 3, 2, or 1 signal. Multiple signals may be of the same type or of different types. Signals of different types may be fluorescent signals with different fluorescent wavelengths. Signals of different types may be generated by detectable labels comprising different fluorophores. Signals of the same type may be of different intensities (e.g., different intensities of the same fluorescent wavelength). Signals of the same type may be signals detectable in the same color channel. Signals of the same type may be generated by detectable labels comprising the same fluorophore. Detectable labels comprising the same fluorophore may generate different signals by nature of being at different concentrations, thereby generating different intensities of the same signal type.

The methods presented in this disclosure may be used with any quantifiable signal. In some cases, this disclosure provides methods to quantify targets using a single component of a signal (e.g., intensity). For example, an analysis may rely on a multiplicity of signal intensity without consideration of color. Although fluorescent probes have been used to illustrate this principle, the disclosed methods are equally applicable to any other method providing a quantifiable signal, including an electrochemical signal and a chemiluminescent signal.

The methods presented in this disclosure may also utilize the measurement of a signal in at least two dimensions, also referred to as the measurement of at least two components of a signal (e.g., color and intensity). In some cases, a quantifiable signal comprises a waveform that has both a frequency (wavelength) and an amplitude (intensity). A signal may be an electromagnetic signal. An electromagnetic signal may be a sound, a radio signal, a microwave signal, an infrared signal, a visible light signal, an ultraviolet light signal, an x-ray signal, or a gamma-ray signal. In some cases, an electromagnetic signal may be a fluorescent signal, for example a fluorescence emission spectrum that may be characterized in terms of wavelength and intensity.

In certain portions of this disclosure, the signal is described and exemplified in terms of a fluorescent signal. This is not meant to be limiting, and one of ordinary skill in the art will readily recognize that the principles applicable to the measurement of a fluorescent signal are also applicable to other signals. For example, like fluorescent signals, any of the electromagnetic signals described above may also be characterized in terms of a wavelength and an intensity. The wavelength of a fluorescent signal may also be described in terms of color. The color may be determined based on measuring intensity at a particular wavelength or range of wavelengths, for example by determining a distribution of fluorescent intensity at different wavelengths and/or by utilizing a band pass filter to determine the fluorescence intensity within a particular range of wavelengths. Intensity may be measured with a photodetector. A range of wavelengths may be referred to as a “channel,” “color channel,” or “optical channel.”

The presence or absence of one or more signals may be detected. One signal may be detected, or multiple signals may be detected. Multiple signals may be detected simultaneously. Alternatively, multiple signals may be detected sequentially. The presence of a signal may be correlated to the presence of a nucleic acid target. The presence of least 1, 2, 3, 4, 5, 6, 7, 8, 9, 10, or more signals may be correlated with the presence of at least one of at least 1, 2, 3, 4, 5, 6, 7, 8, 9, 10, or more nucleic acid targets. The absence of a signal may be correlated with the absence of corresponding nucleic acid targets. The absence of least 1, 2, 3, 4, 5, 6, 7, 8, 9, 10, or more signals may be correlated with the absence of each of at least 1, 2, 3, 4, 5, 6, 7, 8, 9, 10, or more nucleic acid target molecules.

### Kits

The present disclosure also provides kits for multiplex analysis. Kits may comprise one or more oligonucleotide probes. Oligonucleotide probes may be lyophilized. Different oligonucleotide probes may be present at different concentrations in a kit. Oligonucleotide probes may comprise a fluorophore and/or one or more quenchers.

Kits may comprise one or more sets of oligonucleotide primers (or “amplification oligomers”) as described herein. A set of oligonucleotide primers may comprise paired oligonucleotide primers. Paired oligonucleotide primers may comprise a forward oligonucleotide primer and a reverse oligonucleotide primer. A set of oligonucleotide primers may be configured to amplify a nucleic acid sequence corresponding to particular targets. For example, a forward oligonucleotide primer may be configured to hybridize to a first region (e.g., a 3′ end) of a nucleic acid sequence, and a reverse oligonucleotide primer may be configured to hybridize to a second region (e.g., a 5′ end) of the nucleic acid sequence, thereby being configured to amplify the nucleic acid sequence. Different sets of oligonucleotide primers may be configured to amplify nucleic acid sequences. In one example, a first set of oligonucleotide primers may be configured to amplify a first nucleic acid sequence, and a second set of oligonucleotide primers may be configured to amplify a second nucleic acid sequence. Oligonucleotide primers configured to amplify nucleic acid molecules may be used in performing the disclosed methods. In some cases, all of the oligonucleotide primers in a kit are lyophilized.

Kits may comprise one or more nucleic acid enzymes. A nucleic acid enzyme may be a nucleic acid polymerase. A nucleic acid polymerase may be a deoxyribonucleic acid polymerase (DNase). A DNase may be a Taq polymerase or variant thereof. A nucleic acid enzyme may be a ribonucleic acid polymerase (RNase). An RNase may be an RNase III. An RNase III may be Dicer. The nucleic acid enzyme may be an endonuclease. An endonuclease may be an endonuclease I. An endonuclease I may be a T7 endonuclease I. Kits may comprise instructions for using any of the foregoing in the methods described herein.

Additionally, kits may include one or more synthetic (or otherwise generated to be different from the target of interest) primer/probe sets for digital PCR reactions. The one or more synthetic primer/probe sets may be used to calibrate the location of multiple clusters in a multiplex. The kit may also include a known amount of template.

Kits provided herein may be useful in, for example, calculating at least first and second sums, each being a sum of multiple target signals corresponding with a first and second chromosome.

### Systems

Methods as disclosed herein may be performed using a variety of systems. The systems may be configured such the steps of the method may be performed. For example, the systems may comprise a detector for the detection of signals as described elsewhere herein. The system may comprise a processor configured to process, receive, plot, or otherwise represent the data obtained from the detector. The processor may be configured to process the data as described elsewhere herein. The processor may be configured to generate a report of the results obtained from the assay. The results of the assay may be uploaded into a remote server, or other computer systems as described elsewhere herein. The results may be uploaded and sent to a subject's medical provider or an institution monitoring the spread of a disease. The results may also be sent to the subject directly. The subject, medical provider, or other institution may be able to access the remote server such review or analyze the results. For example, the results may then be transmitted to another institution/or medical professional for monitoring or for providing recommendations for the subject. In additional to the data generated for the detection of targets, the data may be used to monitor a geographical location of the assay or subject, for example to allow monitoring of the transmission of a disease. These results can then be uploaded into a cloud database or other remote database for storage and transmission to or access by a variety or individuals and institutions which may use the results of the assay. The results may be obtained on a smart phone or other computer system as disclosed elsewhere herein which may display the results.

The present disclosure provides computer systems that are programmed to implement methods of the disclosure. The computer system can perform various aspects of the present disclosure. The computer system can be an electronic device of a user or a computer system that is remotely located with respect to the electronic device. The electronic device can be a mobile electronic device.

The computer system may include a central processing unit (CPU, also “processor” and “computer processor” herein), which can be a single core or multi core processor, or a plurality of processors for parallel processing. The computer system may include memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. The network, in some cases with the aid of the computer system, can implement a peer-to-peer network, which may enable devices coupled to the computer system to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system can be included in the circuit. In some cases, the circuit is an application specific integrated circuit (ASIC).

The storage unit can store files, such as drivers, libraries and saved programs. The storage unit can store user data, e.g., user preferences and user programs, or raw data or processed results from the assays. The computer system in some cases can include one or more additional data storage units that are external to the computer system, such as located on a remote server that is in communication with the computer system through an intranet or the Internet.

The computer system can communicate with one or more remote computer systems through the network. For instance, the computer system can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system via the network.

The computer system may transmit data associated with or collected during operation of the systems and method described herein to one or more computer systems through a network. The data may be results, conclusions, graphical representations of data, raw outputs, inputs, device or program settings, performance analytics, or other information. The data may be transmitted to another computer system for the purpose of further processing, analyzing, or otherwise transforming the data. The data may then be further transmitted back to the originating computer, to another computer system, or both. The transmission may be physical, such as through a wired network or a physical storage medium, wireless, such as through Bluetooth® or a wireless network connection, or a combination of both, such as a wireless internet transmission. The transmission of the data may be executed manually by a user or through an automated processes, such as programs or algorithms, designed to automatically transmit the data.

The computer system may be accessed by a remote user in order to retrieve data associated with or collected during the operation of the systems and method described herein. The access may be directly to the computer system or through a series of interconnected computer systems. The access and data retrieval may be accomplished manually or automatically by means of a program or algorithms or through a combination of both.

Methods as described herein can be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system can include or be in communication with an electronic display that comprises a user interface (UI) for providing, for example, plots of data, plots of kinetic signatures, information relating to signal amplitude, Examples of Us include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, parameterize data points or fit data point to specified mathematical functions, in order to quantify analytes.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### EXAMPLES

### Example 1—Analytical Performance and Concordance with Next-Generation Sequencing of a Rapid Multiplexed dPCR Panel for the Detection of Actionable DNA and RNA Biomarkers in Non-Small Cell Lung Cancer

### Background:

A highly multiplexed digital PCR (dPCR) assay that detectED 15 relevant non-small cell lung cancer (NSCLC) variants in eight genes using amplitude modulation and multi-spectral encoding was performed. The panel was designed to capture actionable variants in NSCLC, be compatible with formalin-fixed paraffin embedded (FFPE) tissue specimens, and utilizes a low mass input. The use of dPCR for the panel reduced the complexity of the workflow and turnaround time (TAT) by reducing the number of user manipulations in comparison to NGS-based workflows. Cloud based analysis further simplified and accelerated results interpretation, allowing for results generation in less than 24 hours. Furthermore, testing for multiple actionable biomarkers in a single test potentially improved tissue requirements compared to single gene testing. The panel performance included analytical sensitivity, analytical reactivity (inclusivity), and method correlation with current NGS methodologies.

### Materials:

Plasmids containing the target sequences were obtained from IDT (San Diego, CA, USA). FFPE specimens were obtained from Precision for Medicine (Frederick, MD, USA), BioChain (Newark, CA, USA), or CHTN (Durham, NC, USA). Oncomine Precision Assay testing was performed by Precision for Medicine, and results were reported for all positive specimens. Negative specimens, non-tumor adjacent tissue, were reported negative by pathology. The mean age of the individual at time of sample acquisition was 63.5 years (standard deviation of 11.1).

### Methods:

High Definition (HDPCR) NSCLC panel utilizes dPCR, where endpoint fluorescent intensities are modulated such that each unique target produces a unique endpoint intensity. The HDPCR NSCLC Panel consisted of three wells; two wells detect DNA targets, and one well to detect RNA fusions. All runs were performed on the QIAcuity® (Qiagen, Germany) using the QIAcuity Nanoplate 26K 24-well plate. The master mix for DNA wells was formulated by combining 10.5 μL of QIAcuity Probe Master Mix, 8.4 μL of HDPCR Mix and 2.1 μL of molecular grade water per reaction. The master mix for each RNA well was formulated by combining 10.5 μL of QIAcuity OneStep Advance Probe Master Mix (Qiagen, Germany), 0.45 μL of OneStep RT Mix (Qiagen, Germany), 8.4 μL of HDPCR Mix, and 1.68 μL of molecular grade water per reaction. After preparation of the master mix, 21 μL of the sample was added to 21 μL of the appropriate master mix and mixed thoroughly. From this mixture, 39 μL was added to a well on the QIAcuity Nanoplate. The plate then underwent thermocycling on the QIAcuity according to the instructions for use. Analysis was carried out using a cloud-based analysis platform, which reported out detected targets and Mutant Allele Fraction (MAF). The MAF is calculated as (target counts/IC counts)×100.

The cloud based analysis system was designed to integrate analytical and mathematic processing designed to enhance the processing and analysis of dPCR based information. The methods and systems described herein were integrated into the cloud based analysis platform for the analysis of the absolute quantification of the target counts for each unique target.

### Analytical Sensitivity:

Negative FFPE background was prepared by extracting DNA and RNA from pathology-negative FFPE samples using the Maxwell HT FFPE DNA Isolation System. The limit of detection for DNA targets was established by spiking plasmids containing the target sequences into negative FFPE background at various MAF concentrations. The RNA fusion targets were transcribed from plasmids using the HiScribe T7 High Yield RNA Synthesis Kit (NEB, Ipswich, MA), isolated using the Monarch Kit (NEB, Ipswich, MA) and spiked into negative FFPE RNA background. The limit of detection for DNA targets was established at two input amounts; 20 ng and 7.5 ng of DNA per well (40 ng and 15 ng in total for both DNA wells). The RNA fusion targets were tested at 5 ng total RNA input. Range-finding was conducted by testing decreasing serial dilutions. For each target, the lowest concentration at which all replicates were positive during range-finding was evaluated with 20 replicates. The limit of detection is reported at the lowest concentration, where greater than 18/20 replicates were detected.

### Analytical Inclusivity:

In-silico analysis of designs was performed using the COSMIC Mutation Database. After filtering, prevalence was calculated based on the count number of distinct entries in the “sample name field”. Analytical inclusivity was evaluated for the HDPCR NSCLC Panel by spiking in quantified plasmids containing the different sequences into the appropriate negative FFPE background (RNA or DNA, depending on the well). Each plasmid was tested at 3-5× the limit of detection in three replicates. If no replicate was detected, the plasmid was tested at 10× higher concentration. If any replicate was negative at the higher concentration, the assay was determined to be not inclusive for the specific sequence. Prevalence was estimated from the reported occurrences of unique Sample ids for each COSMIC ID (LEGACY_MUTATION_ID) associated with a reportable in the filtered COSMIC Mutation Database.

### Concordance Study:

106 unique FFPE blocks (77 positive samples with the Oncomine Precision Assay and 29 pathology negative samples) from lung tissue were enrolled in the study. DNA and RNA were extracted from a single 10 μm curl using the Maxwell HT FFPE DNA Isolation System (Promega, Madison, WI) on the KingFisher™ Flex instrument (Thermofisher, Carlsbad, CA). Following extraction, eluates were quantified using the Qubit dsDNA BR Assay Kit (Invitrogen, Waltham MA) or the Qubit RNA BR Assay Kit (Invitrogen, Waltham, MA). Samples were evaluated with the HDPCR NSCLC Panel according to the above mentioned methods. Results from the Oncomine Precision Assay (from separate sections of the same block) and the HDPCR NSCLC Panel was compared, and any discordant samples (same section as evaluated by HDPCR) were sent for discordant resolution using the Variant Plex solid tumor focus (Archer, Boulder, CO) or the Fusion Plex Lung (Archer, Boulder, CO). Results from discordant analysis were then detailed for each target.

### Results:

### Analytical Sensitivity, Limit of Detection (LOD):

The analytical sensitivity is reported in estimated Mutant Allele Fraction (MAF). Each assay features an internal control (IC) to determine if sufficient amplifiable nucleic acid has been loaded into the well. At 20 ng input (1854 average IC Counts) DNA per well, the LOD ranged from 0.8% to 4.9% MAF (Table 1). When the total DNA input was decreased to 7.5 ng per well (476 average IC Counts), the limit of detection ranged from 2.4% to 10.9% MAF (Table 1). With an input of 5 ng (97 average IC counts), the limit of detection for RNA targets ranged from 24 to 150 counts (Table 2). These results indicate that even with minimal inputs of DNA and RNA, the HDPCR NSCLC panel is sensitive for all targets.

TABLE 1

| | 7.5 ng Input | | | 20 ng Input | | |
|---|---|---|---|---|---|---|
| | Mean | Detection | | Mean | Detection | |
| Target | Counts | Rate | MAF | Counts | Rate | MAF |
| EGFR exon 19 deletions | 76.7 | 20/20 | 10.9% | 30.2 | 20/20 | 1.2% |
| EGFR G719X | 40.9 | 20/20 | 6.2% | 33.9 | 20/20 | 2.7% |
| EGFR S768I | 51.0 | 20/20 | 9.6% | 67.2 | 20/20 | 3.2% |
| ERBB2 insertions | 41.6 | 20/20 | 7.3% | 32.2 | 20/20 | 2.4% |
| EGFR L858R | 71.5 | 20/20 | 8.8% | 56.0 | 19/20 | 2.6% |
| EGFR exon 20 | 60.4 | 20/20 | 10.1% | 58.3 | 20/20 | 4.9% |
| insertions | | | | | | |
| BRAF V600E | 21.0 | 20/20 | 2.4% | 45.1 | 20/20 | 2.2% |
| KRAS G12C | 22.5 | 20/20 | 3.6% | 34.2 | 20/20 | 2.3% |
| EGFR T790M | 37.3 | 20/20 | 9.5% | 19.5 | 20/20 | 0.8% |
| EGFR L861Q | 40.5 | 20/20 | 8.3% | 40.7 | 20/20 | 3.5% |

TABLE 2

5 ng Input

| | Mean | Detection |
|---|---|---|
| Target | Counts | Rate |
| ALK fusions | 25.1 | 19/20 |
| RET fusions | 23.6 | 20/20 |
| ROSI fusions | 60.8 | 20/20 |
| MET Exon 14 skipping mutations | 149.5 | 20/20 |
| NTRK 1/2/3 fusions | 48.4 | 20/20 |

### Inclusivity:

Inclusivity was evaluated both in silico for DNA targets, and empirically, for DNA and RNA fusion targets, by testing plasmids spiked in FFPE negative matrix. The in-silico analysis resulted in 31 DNA targets flagged for empirical evaluation. The results for in silico and empirical analysis for DNA targets are reported in Table 3. RNA fusion targets were all evaluated empirically, with 96 different variations evaluated. The results are reported in Table 4.

TABLE 3

| | | | | | | | Prevalence per 1000 | |
|---|---|---|---|---|---|---|---|---|
| | | | | | | In Silico + | Positive | Missed |
| | In Silico Analysis | | | | | Empirical | Samples | calls dues |
| | | Not | | Empirical Analysis | | Prevalence | per | to |
| Target | Evaluated | Inclusive | Inclusive | Evaluated | Detected | Coverage | 1000 | inclusivity |
| EGFR | 24 | 5 | 19 | 8 | 8 | 89.2% | 9 | 1 |
| Exon 20 | | | | | | | | |
| Insertions | | | | | | | | |
| EGFR | 23 | 2 | 13 | 16 | 14 | 98.6% | 131 | 1.8 |
| Exon 19 | | | | | | | | |
| Deletions | | | | | | | | |
| EGFR | 3 | 3 | 3 | 3 | 3 | 100% | 10 | 0 |
| G719X | | | | | | | | |
| ERBB2 | 6 | 1 | 5 | 5 | 5 | 99.4% | 9 | <0.1 |

TABLE 4

| | | | Overall | | Missed |
|---|---|---|---|---|---|
| | | | Inclusivity | | calls |
| | | | (Prevalence | Positive | dues to |
| Target | Evaluated | Detected | Weighted) | Patients/1000 | inclusivity |
| ALK | 41 | 40 | 99% | 27 | <0.3 |
| ROS1 | 25 | 25 | 100% | 4 | 0 |
| RET | 20 | 20 | 98.7% | 4 | <0.1 |
| NTRK | 10 | 10 | 95.7% | 0 | 0 |

### Concordance Study:

The HDPCR NSCLC panel was used to evaluate 106 unique FFPE samples. The IC in DNA wells failed in 15 of the 106 samples, while the RNA IC failed in 6 of the 106 samples. The failure of the IC in samples was correlated with the source vendor, which ranged from 0% to 63% internal control failures. In samples where the IC passed, the concordance of the HDPCR NSCLC panel with the comparator method was 97.8% (1399/1430). The positive percent agreement (PPA) for individual targets ranged from 50.0%-100.0% and the positive predictive value (PPV) ranged from 62.5 to 100.0% before discordant resolution, low values are driven by the low number of positives samples available for some targets and high level of discordant results. The negative percent agreement (NPA) ranged from 97.0%-100.0% and the negative predicted value (NPV) ranged from 94.3%-100%. Discordant samples were evaluated, from the same extraction, if possible, with either the VariantPlex solid tumor focus panel for DNA targets or the FusionPlex Lung for RNA targets. After discordant resolution, each target's PPA ranged from 71.4%-100.0% and PPV ranged from 71.4%-100.0%. The NPA ranged from 97.9%-100% and the NPV ranged from 97.9%-100% for individual targets after discordant resolution. In total, there were 31 discordant results with the comparator method. Discordant resolution agreed with the HDPCR NSCLC Panel in 71.0% (22/31) of discordant results and aligned with the comparator in 29.0% (9/31) of results. Of the nine discordant results that aligned with the comparator method, 44.4% (4/9) of the results were novel fusions that are outside the inclusivity of the HDPCR NSCLC Panel, with 5/9 incongruous samples remaining.

### Conclusion:

The HDPCR NSCLC Panel uses a more simplified workflow involving only 2 touch points post extraction on dPCR, allowing a TAT of less than 4 hours excluding extraction time. The results from the studies presented here illustrated that the HDPCR NSCLC Panel utilizing dPCR achieved sensitivity down to 0.8% MAF and greater than 99% accuracy with comparator results after discordant resolution. Lung samples are often characterized by limited tissue availability for molecular testing. Collection methods for specimens in NSCLC include fine needle aspirates (FNA), core needle biopsy (CNB), and resected tissue, offering varying amounts of surveyable genetic material. dPCR has been demonstrated to have sensitivity with low DNA input amounts but is limited by the scope of the variants examined in a single well. Utilizing the systems and method described herein enabled the absolute quantification of these samples with minimal impact to either the sensitivity or inclusivity. Here a MAF limit of detection between 2.4-10.9% at as little as 15 ng of DNA input split across two wells (7.5 ng per well) was demonstrated. While at high DNA input amounts, a MAF limit of detection between 0.8-4.9% at 40 ng input (20 ng per well) was reported. One disadvantage of traditional PCR approaches to the detection of variants is poor sequence coverage or inclusivity. The HDPCR NSCLC Panel was designed for high inclusivity for highly variable targets like EGFR exon 20 insertions (89%), EGFR exon 19 deletions (95%), and RNA fusions (95-100%). The high inclusivity provides increased confidence in negative results. The HDPCR NSCLC Panel demonstrated high concordance (>97%) with the comparator methods. Discordant results, 71.0% (22/31), between the comparator and the HDPCR NSCLC panel resolved in favor of HDPCR. Taken together, the results demonstrate how the HDPCR NSCLC Panel can test for actionable variants with low nucleic acid input using a simple PCR-based workflow. For the RNA fusion targets, the primary source of false negatives, five of nine, was due to the detection of novel fusions that are not within the scope of the HDPCR panel. The ability to provide faster, accurate results for actionable biomarkers is a key step toward democratizing testing. Here a dPCR panel that provides a coverage of actionable biomarkers with high concordance with NGS was presented. The simplified workflow and analysis make it a potential solution for improving accessibility to relevant biomarker testing.

### Example 2—Analysis of Multiple Loci on a Single Chromosome Using Digital PCR

FIGS. 4A-4B show the results of a digital PCR simulation of 3 targets with individual intensity units of “1”, with multiple targets being present results in the peaks at higher intensity levels. The simulation consisted of developing 20,000 virtual partitions and randomly assigning each input DNA copy to a partition. For each partition, if a particular ‘target’ DNA was present after random assignment, an intensity level of “1” was added to the intensity level of the droplet. Thus, if the partition had no DNA, an intensity of “0” was assigned; if there were one or more copies of DNA of only one target, an intensity of “1” was assigned; if there were one or more copies of DNA of two targets, an intensity of “2” was assigned; and if there were one or more copies of all three targets, an intensity level of “3” was assigned. A small, normally distributed amount of noise was added to the intensity for each droplet for east of plotting. In this example, a ‘target’ refers to each individual intensity-generating probe sequence, however in other cases a target may refer to a larger collection of targets, for example multiple loci on an individual chromosome.

FIGS. 5A-5B show a similar simulation of a digital PCR assay for 10 different loci from a single chromosome. Ten targets at an intensity level of 1 were present and an input copy of 10,000 copies per target were distributed among 20,000 partitions, resulting in partitions that have up to ten different loci present in any given droplet. A clear and distinct pattern emerged, allowing the calculation of the initial input quantity and 100,000 total counts of positive DNA input.

### Example 3—Absolute Quantification of Two Targets Using Digital PCR with a Poisson Model

A single sample containing at least one nucleic acid target sequence, at least one amplification oligomer, at least one detection oligonucleotide, dNTPs, a thermostable DNA polymerase, and other PCR reagents is partitioned into approximately 20,000 evenly sized partitions. Following partitioning, each partition is then subject to end-point PCR. Partitions emitting a fluorescent signal are marked “positive” and scored as “1,” and partitions without detectable fluorescence are deemed “negative” and scored as “0.” A Poisson statistical model is then applied to the data to calculate the probability of a given partition receiving zero, one, two, three or more copies. This “correction” is then applied to the data thus enabling all molecules in the starting sample to be accounted for, yielding absolute quantification.

### Example 4—Absolute Quantification of Three Targets Using Digital PCR with a Poisson Model

The experiment of example 2 is repeated with a sample containing 3 targets. The signals generated by the plurality of partitions are such that some partitions positive for two or more targets, could be representative of multiple target combinations (i.e, the first and second target or the third target). The concentration of target 1 and 2 is calculated using only partitions positive for only target 1 or 2 respectively through statistical analysis discussed above. However, the same strategy cannot be used for target 3 since an unknown number of partitions are positive for target 3 due to the ambiguity discussed above. However, by applying a statistical model and using the equation:

$${λ}_{3} = - ln ( 1 - {p}_{3} ) = - ln ( 1 - \frac{{N}_{t 3}}{{N}_{0} + {N}_{t 3}} ) = - ln ( 1 - \frac{{N}_{0} {N}_{3} - {N}_{1} {N}_{2}}{{{N}_{0}}^{2} + {N}_{0} {N}_{3} - {N}_{1} {N}_{2}} )$$

In which Nt3 is the number of partitions positive for target 3 only and
N0: Number of partitions with no targets
N1: Number of partitions with target 1
N2: Number of partitions with target 2
N3: Number of partitions with both target 1 and target 2 OR only target 3 (i.e., ambiguous).
   The concentration of target 3 is estimated directly from only partitions positive for zero or one targets while the true count of partitions positive for any combination of targets (e.g. Nt1+t2) is unknown.

### Example 5—Absolute Quantification of Two Targets with Using Digital PCR without Multiply Positive Partitions

The experiment of example 2 is repeated with two targets in the sample. Once data is received from the digital PCR run and identification is performed, the partition counts are used to calculate target concentrations using the equations:

$${λ}_{1} = - ln ( 1 - {p}_{1} ) = - ln ( 1 - \frac{{N}_{1}}{{N}_{0} + {N}_{1}} )$$

$$and$$

$${λ}_{2} = - ln ( 1 - {p}_{2} ) = - ln ( 1 - \frac{{N}_{2}}{{N}_{0} + {N}_{2}} )$$

where the quantity of nucleic acid is calculated in terms of average number of copies per partition (λ) for each target and
N0: Number of partitions with no targets;
N1: Number of partitions with target 1;
N2: Number of partitions with target 2;

Thus, the calculation for both target 1 and target 2 are performed without the number of partitions with both target 1 and target 2.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method of quantifying a first nucleic acid target and a second nucleic acid target in a sample, the method comprising:
(a) providing a mixture comprising:
i. said first nucleic acid target;
ii. said second nucleic acid target;
iii. a first plurality of oligonucleotide probes, wherein the first plurality of oligonucleotide probes hybridizes to said first nucleic acid target; and
iv. a second plurality of oligonucleotide probes, wherein the second plurality of oligonucleotide probes hybridizes to said second nucleic acid target;
(b) partitioning said mixture into a plurality of partitions;
(c) in said plurality of partitions, generating a plurality of signals from said first plurality of probes and said second plurality of probes, wherein said plurality of signals are detectable in one or more color channels;
(d) detecting from multiple partitions of said plurality of partitions said plurality of signals in said one or more color channels; and
(e) based on said detecting, quantifying said first nucleic acid target and said second nucleic acid target in said sample wherein such quantification does not comprise using a determined number of partitions positive for two or more of said targets.

2. The method of claim 1, wherein said partitioning generates at least 10 partitions positive for two or more of said targets.

3. The method of claim 1, wherein said partitioning generates at least 100 partitions positive for two or more of said targets.

4. The method of claim 1, wherein said partitioning generates a number of partitions positive for two or more of said targets that is greater than 10% of the total number of partitions.

5. The method of claim 1, wherein said partitioning generates a number of partitions positive for two or more of said targets that is greater than 20% of the total number of partitions.

6.-8. (canceled)

9. The method of claim 1, wherein said plurality of signals is generated by excitation of said fluorophore.

10.-21. (canceled)

22. The method of claim 1, wherein said first nucleic acid target or second nucleic acid target comprises a chromosomal nucleic acid sequence.

23. The method of claim 22, wherein said chromosomal nucleic acid sequence is chosen from among the nucleic acid sequences of chromosome 21, chromosome 18, chromosome 15, chromosome 13, an X chromosome, or a Y chromosome.

24. The method of claim 1, wherein said first nucleic acid target comprises a first chromosomal nucleic acid sequence and said second nucleic acid target comprises a second chromosomal nucleic acid sequence.

25.-41. (canceled)

42. A method of quantifying at least a first nucleic acid in a target in a sample, the method comprising:
(a) providing a mixture comprising:
i. a first target nucleic acid target;
ii. a second target nucleic acid target;
iii. a third target nucleic acid target;
iv. a first plurality of oligonucleotide probes, wherein the first plurality of oligonucleotide probes hybridizes to said first nucleic acid target; and
v. a second plurality of oligonucleotide probes, wherein the second plurality of oligonucleotide probes hybridizes to said second nucleic acid target; and
vi. a third plurality of oligonucleotide probes, wherein the third plurality of oligonucleotide probes hybridizes to said third nucleic acid target; and
(b) partitioning said mixture into a plurality of partitions;
(c) in said plurality of partitions, generating a plurality of signals from said first plurality of probes, said second plurality of probes and said third plurality of probes, wherein said plurality of signals are detectable in one or more color channels;
(d) detecting from multiple partitions of said plurality of partitions said plurality of signals in one or more color channels,
wherein said plurality of signals comprise an indifferentiable signal from at least one partition of the plurality of partitions which may comprise said first target nucleic acid target, said second target nucleic acid target, said third target nucleic acid target, or a combination thereof; and
(e) based on said detecting, quantifying said first nucleic acid target in said sample wherein such quantification does not comprise using a determined number of partitions positive for said indifferentiable signal generated from said two or more other targets.

43. The method of claim 42, wherein said partitioning generates at least 10 partitions generating an indifferentiable signal.

44. The method of claim 42, wherein said partitioning generates at least 100 generating an indifferentiable signal.

45. The method of claim 42, wherein said partitioning generates a number of partitions generating an indifferentiable signal is greater than 10% of the total number of partitions.

46. The method of claim 42, wherein said partitioning generates a partitions generating an indifferentiable signal is greater than 20% of the total number of partitions.

47. The method of claim 42, wherein the first, second, and/or third plurality of probes comprises a fluorophore.

48. The method of claim 47, wherein said plurality of signals is generated by excitation of said fluorophore.

49. The method of claim 42, wherein said plurality of signals is generated by degradation of said first, second, and/or third plurality of oligonucleotide probes.

50. The method of claim 42, wherein (c) comprises amplifying said plurality of nucleic acid molecules, thereby generating said plurality of signals.

51. The method of claim 50, wherein said amplification comprises a polymerase chain reaction.

52. The method of claim 50, wherein said amplification comprises an isothermal amplification reaction.

53.-91. (canceled)
